SEQUENCE LISTING (10 sequences)

SEQ ID NO: 1 - protein, 118 aa, Artificial Sequence
  VX15/2503 VH
QVQLVQSGAEVKKPGSSVKVSCKASGYSFSDYYMHWVRQAPGQGLEWMGQINPTTGGASYNQKFKGKATITVDKSTSTAYMELSSLRSEDTAVYYCARYYYGRHFDVWGQGTTVTVSS

SEQ ID NO: 2 - protein, 10 aa, Artificial Sequence
  VX15/2503 HCDR1
GYSFSDYYMH

SEQ ID NO: 3 - protein, 17 aa, Artificial Sequence
  VX15/2503 HCDR2
QINPTTGGASYNQKFKG

SEQ ID NO: 4 - protein, 9 aa, Artificial Sequence
  VX15/2503 HCDR3
YYYGRHFDV

SEQ ID NO: 5 - protein, 111 aa, Artificial Sequence
  VX15/2503 VL
DIVMTQSPDSLAVSLGERATINCKASQSVDYDGDSYMNWYQQKPGQPPKLLIYAASNLESGVPDRFSGSGSGTDFTLTISSLQAEDVAVYYCQQSNEDPYTFGQGTKLEIK

SEQ ID NO: 6 - protein, 15 aa, Artificial Sequence
  VX15/2503 LCDR1
KASQSVDYDGDSYMN

SEQ ID NO: 7 - protein, 7 aa, Artificial Sequence
  VX15/2503 LCDR2
AASNLES

SEQ ID NO: 8 - protein, 9 aa, Artificial Sequence
  VX15/2503 LCDR3
QQSNEDPYT

SEQ ID NO: 9 - protein, 118 aa, Artificial Sequence
  Mab 67 VH
QVQLQQSGPELVKPGASVKISCKASGYSFSDYYMHWVKQSPENSLEWIGQINPTTGGASYNQKFKGKATLTVDKSSSTAYMQLKSLTSEESAVYYCTRYYYGRHFDVWGQGTTVTVSS

SEQ ID NO: 10 - protein, 111 aa, Artificial Sequence
  Mab 67 VL
DIVMTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

(19) United States
(12) Patent Application Publication
(10) Pub. No.: US 2019/0383836 A1
(43) Pub. Date: Dec. 19, 2019

(54) EARLY DETECTION OF GLIAL CELL ACTIVATION IN NEURODEGENERATIVE OR NEUROINFLAMMATORY DISEASES

(71) Applicant: Vaccinex, Inc., Rochester, NY (US)

(72) Inventor: Maurice ZAUDERER, Pittsford, NY (US)

(73) Assignee: Vaccinex, Inc., Rochester, NY (US)

(21) Appl. No.: 16/488,123

(22) PCT Filed: Feb. 20, 2018
(86) PCT No.: PCT/US2018/018794

§ 371 Date: Aug. 22, 2019

Related U.S. Application Data
(60) Provisional application No. 62/461,945, filed on Feb. 22, 2017.

(51) Int. Cl. G01N 33/68; G01N 33/66; C07K 16/28; A61P 25/28; A61P 25/16

(52) U.S. Cl. CPC G01N 33/6896; G01N 33/66; G01N 33/6857; C07K 16/2803; A61P 25/28; A61K 2039/505; G01N 2800/2821; G01N 2800/2835; G01N 2800/52; C07K 2317/76; A61P 25/16

(57) ABSTRACT

The disclosure provides a method for determining the efficacy of treatment with a SEMA4D antagonist, e.g., a SEMA4D antagonist antibody in the treatment of a neuroinflammatory or neurodegenerative disease, disorder, or injury, where the method provides differential measurement of glucose uptake in the brain, e.g., by FDG-PET imaging.

### CROSS-REFERENCE TO RELATED APPLICATIONS

This application claims the benefit of U.S. Provisional Patent Application Ser. No. 62/461,945, filed Feb. 22, 2017, which is incorporated herein by reference in its entirety.

### SEQUENCE LISTING STATEMENT

A sequence listing containing the file named 58008_172847_Seq List_ST25.txt which is 5880 bytes (measured in MS-Windows®) and created on Feb. 20, 2018, comprises 10 sequences, is provided herewith via the USPTO's EFS system, and is incorporated herein by reference in its entirety.

### BACKGROUND

Semaphorin 4D (SEMA4D), also known as CD100, is a transmembrane protein that belongs to the semaphorin gene family. SEMA4D is expressed on the cell surface as a homodimer, but upon cell activation SEMA4D can be released from the cell surface via proteolytic cleavage to generate sSEMA4D, a soluble form of the protein, which is also biologically active. See Suzuki et al., Nature Rev. Immunol. 3:159-167 (2003); Kikutani et al., Nature Immunol. 9:17-23 (2008).

SEMA4D is expressed at high levels in lymphoid organs, including the spleen, thymus, and lymph nodes, and in non-lymphoid organs, such as the brain, heart, and kidney. In lymphoid organs, SEMA4D is abundantly expressed on resting T cells but only weakly expressed on resting B cells and antigen-presenting cells (APCs), such as dendritic cells (DCs). Its expression, however, is upregulated in these cells following activation by various immunological stimuli. The release of soluble SEMA4D from immune cells is also increased by cell activation.

SEMA4D is implicated in neurodegenerative disorders, autoimmune diseases, demyelinating diseases, and cancer. In the central nervous system (CNS), SEMA4D is expressed on, e.g., infiltrating immune cells and oligodendrocyte precursor cells and its receptors are expressed on, e.g., neurons, oligodendrocytes, astrocytes, and endothelial cells (Okuno, T., et al., J. Immunol. 184:1499-1506 (2010)). SEMA4D can serve as an axonal guidance molecule (Swiercz et al., Neuron 35:51-63 (2002)) and can mediate GABAergic and glutamatergic synapse development (Paradis et al., Neuron 53:217-232 (2007)) among other activities.

SEMA4D has also been shown to play a role in the migration and differentiation of neuronal and oligodendrocyte precursor cells, CNS inflammation, and neurodegeneration. For example, SEMA4D-deficient mice are resistant to the development of experimental autoimmune encephalomyelitis (EAE) (Kumanogoh A et al., Immunity 13:621-631 (2000)), and blockade of SEMA4D can inhibit microglial activation and neuroinflammation in EAE (Okuno, T., et al., J. Immunol. 184:1499-1506 (2010)). Similarly, SEMA4D stimulation of endothelial cells can lead to production of the proinflammatory cytokine IL-8 (Yang, Y H et al., PLoS One 6:e25826 (2011)).

Huntington's Disease (HD) is an inherited, fatal neurodegenerative disease resulting from the pathogenic expansion of a polyglutamine-encoding CAG tract in exon 1 of the huntingtin (HTT) gene to 36 or more repeats (Huntington's Disease Collaborative Research Group (HDCRG) Cell 72:971-983 (1993)). The 36 or more glutamines (expanded polyQ) in HTT results in the production of an altered form, called mHTT (mutant HTT) which increases the rate of degeneration of certain types of neurons. The extent of the degeneration is related to poly Q length, and accounts for about 60% of the variation of the age at onset and the rate of progression of symptoms of HD. HD is an autosomal dominant, genetic disorder whereby each person whose parent has HD is born with a 50/50 chance (at risk) of inheriting the single mutated huntingtin gene (via either the X or Y sex chromosomes on which it is carried). Anyone who inherits this gene will, at some point in life, develop the disease. According to the US National Institute of Neurological Disorders and Stroke (NINDS) there are 30,000 US patients suffering from HD at any time, and another 150,000 individuals having a 50% risk of developing the disease. HD is a complex and severely debilitating terminal disease, for which there is no cure.

HD is characterized by motor and cognitive deficits and psychiatric disturbance with death usually occurring 15-20 years after onset. While disease onset, which is clinically defined as presentation of motor deficits, typically occurs in mid-life, many features of HD can present years to decades earlier, including, e.g., immune activation (Bjorkqvist M et al., J. Exp. Med 205:1869-1877 (2008)), striatal atrophy and loss of brain white matter (Tabrizi S J et al., Lancet Neurol. 8:791-801 (2009)). Additionally, severely reduced turnover of cells of the neuronal and oligodendrocyte lineage within the human HD striatum can occur (Ernst A et al., Cell 156:1072-1083 (2014)). Transcriptional dysregulation can be an early feature of HD; for example, expression of SEMA4D and its major CNS receptor, Plexin-B1, can be elevated in the HD striatum and cortex, but not cerebellum (Hodges et al., Hum. Mol. Genet. 15:965-977 (2006)).

Inhibition of SEMA4D signaling through anti-SEMA4D treatment represents a novel approach to therapy for HD. In particular aspects, work with the YAC128 transgenic mouse model of HD demonstrated that treatment with an anti-SEMA4D antibody ameliorated certain neuropathological effects, including striatal atrophy, cortical atrophy, and corpus callosum atrophy, and prevented testicular degeneration. Also, a subset of behavioral symptoms was improved in YAC128 mice treated with an anti-SEMA4D antibody, including reduced anxiety-like behavior and rescue of cognitive deficits. See Southwell A L, et al. Neurobiol. Dis. 76:46-56 (2015) and U.S. Pat. No. 9,249,227, granted Feb. 2, 2016. Given the long time periods involved in the development of measurable HD symptoms and alleviation of those symptoms, there remains a need for a method of early, reproducible detection of whether a given therapy, in particular, anti-SEMA4D treatment will be effective in individuals genetically predisposed to develop HD.

### SUMMARY

This disclosure provides a method for determining whether a semaphorin 4D (SEMA4D) antagonist antibody or antigen-binding fragment thereof could be effective in treating a neurodegenerative or neuroinflammatory disease, disorder, or injury.

In one aspect the method includes administering an effective amount of a SEMA4D antagonist antibody or antigen-binding fragment thereof to a subject having, suspected of having, or at risk of developing a neurodegenerative or neuroinflammatory disease, disorder, or injury; measuring the level of glucose uptake in the subject's brain relative to a baseline level of glucose uptake in the subject's brain measured prior to administration of the SEMA4D antagonist; and continuing administration of the SEMA4D antagonist antibody or fragment thereof if an increase in glucose uptake over baseline is detected; or discontinuing administration of the SEMA4D antagonist antibody or fragment thereof if no change or a decrease in glucose uptake relative to baseline is detected.

In another aspect the method includes measuring the baseline level of glucose uptake in the brain of a subject having, suspected of having, or at risk of developing a neurodegenerative or neuroinflammatory disease, disorder, or injury; administering an effective amount of a SEMA4D antagonist antibody or antigen-binding fragment thereof to the subject; remeasuring the level of glucose uptake in the subject's brain following administration of the SEMA4D antagonist antibody or fragment thereof; and continuing administration of the SEMA4D antagonist antibody or fragment thereof if an increase in glucose uptake over baseline is detected; or discontinuing administration of the SEMA4D antagonist antibody or fragment thereof if no change or a decrease in glucose uptake relative to baseline is detected.

In another aspect the method includes administering a starting dose of a SEMA4D antagonist antibody or antigen-binding fragment thereof to a subject having, suspected of having, or at risk of developing a neurodegenerative or neuroinflammatory disease, disorder, or injury; measuring the level of glucose uptake in the subject's brain relative to a baseline level of glucose uptake in the subject's brain measured prior to administration of the SEMA4D antagonist; and adjusting the dose of the SEMA4D antagonist antibody or fragment thereof if an increase in glucose uptake over baseline is detected, the adjustment determined on the level of increase, or discontinuing administration of the SEMA4D antagonist antibody or fragment thereof if no change or a decrease in glucose uptake relative to baseline is detected. According to this aspect the method can further include increasing the dose of SEMA4D antagonist antibody relative to that tested in step (b) and remeasuring the change in level of glucose uptake relative to a new baseline in a different previously untreated patient or in the same patient following withdrawal of treatment in the same patient for a period of time determined to allow accumulation of a historical deficit in that neurodegenerative or neuroinflammatory disease, disorder, or injury, and further adjusting the dose of the SEMA4D antagonist antibody if a further increase is detected.

In another aspect the method includes measuring the baseline level of glucose uptake in the brain of a subject having, suspected of having, or at risk of developing a neurodegenerative or neuroinflammatory disease, disorder, or injury; administering a starting dose of a SEMA4D antagonist antibody or antigen-binding fragment thereof to the subject; remeasuring the level of glucose uptake in the subject's brain following administration of the SEMA4D antagonist antibody or fragment thereof; and adjusting the dose of the SEMA4D antagonist antibody or fragment thereof if an increase in glucose uptake over baseline is detected, the adjustment determined on the level of increase, or discontinuing administration of the SEMA4D antagonist antibody or fragment thereof if no change or a decrease in glucose uptake relative to baseline is detected. According to this aspect the method can further include increasing the dose of SEMA4D antagonist antibody relative to that tested in step (c) and remeasuring the change in level of glucose uptake relative to a new baseline in a different previously untreated patient or in the same patient following withdrawal of treatment in the same patient for a period of time determined to allow accumulation of a historical deficit in that neurodegenerative or neuroinflammatory disease, disorder, or injury, and further adjusting the dose of the SEMA4D antagonist antibody if a further increase is detected.

In another aspect the method includes administering a SEMA4D antagonist antibody or antigen-binding fragment thereof to a subject having, suspected of having, or at risk of developing a neurodegenerative or neuroinflammatory disease, disorder, or injury; measuring the level of glucose uptake in the subject's brain relative to a baseline level of glucose uptake in the subject's brain measured prior to administration of SEMA4D antagonist; and continuing administration of the SEMA4D antagonist antibody or fragment thereof if an increase in glucose uptake over baseline is detected; or discontinuing administration of the SEMA4D antagonist antibody or fragment thereof if no change or a decrease in glucose uptake relative to baseline is detected.

In another aspect the method includes measuring the baseline level of glucose uptake in the brain of a subject having, determined to have, or suspected of having a neurodegenerative or neuroinflammatory disease, disorder, or injury; administering a SEMA4D antagonist antibody or antigen-binding fragment thereof to the subject; remeasuring the level of glucose uptake in the subject's brain following administration of the SEMA4D antagonist antibody or fragment thereof; and continuing administration of the SEMA4D antagonist antibody or fragment thereof if an increase in glucose uptake over baseline is detected; or discontinuing administration of the SEMA4D antagonist antibody or fragment thereof if no change or a decrease in glucose uptake relative to baseline is detected.

In another aspect the method includes administering a SEMA4D antagonist antibody or antigen-binding fragment thereof to a subject having, suspected of having, or at risk of developing a neurodegenerative or neuroinflammatory disease, disorder, or injury; ordering the measurement of the level of glucose uptake in the subject's brain relative to a baseline level of glucose uptake in the subject's brain measured prior to administration of the SEMA4D antagonist; and continuing administration of the SEMA4D antagonist antibody or fragment thereof if an increase in glucose uptake over baseline is detected; or discontinuing administration of the SEMA4D antagonist antibody or fragment thereof if no change or a decrease in glucose uptake relative to baseline is detected.

In another aspect the method includes ordering the measurement of a baseline level of glucose uptake in the brain of a subject having, suspected of having, or at risk of developing a neurodegenerative or neuroinflammatory disease, disorder, or injury; administering a SEMA4D antagonist antibody or antigen-binding fragment thereof to the subject; ordering remeasurement of the level of glucose uptake in the subject's brain following administration of the SEMA4D antagonist antibody or fragment thereof; and continuing administration of the SEMA4D antagonist antibody or fragment thereof if an increase in glucose uptake over baseline is detected; or discontinuing administration of the SEMA4D antagonist antibody or fragment thereof if no change or a decrease in glucose uptake relative to baseline is detected.

In another aspect the method includes measuring the baseline level of glucose uptake in the brain of a subject presented as having, suspected of having, or at risk of developing a neurodegenerative or neuroinflammatory disease, disorder, or injury; and remeasuring the level of glucose uptake in the subject's brain following administration or a SEMA4D antagonist antibody or antigen-binding fragment thereof to the subject by a healthcare provider; and instructing the healthcare provider to continue administration of the SEMA4D antagonist antibody or fragment thereof if an increase in glucose uptake over baseline is detected; or instructing the healthcare provider to discontinue administration of the SEMA4D antagonist antibody or fragment thereof if no change or a decrease in glucose uptake relative to baseline is detected.

In certain aspects, the SEMA4D antagonist antibody or fragment thereof for use in the provided method inhibits SEMA4D interaction with its receptor, e.g., Plexin-B1, Plexin-B2, or CD72. In certain aspects the SEMA4D antagonist antibody or fragment thereof inhibits SEMA4D-mediated Plexin-B1 signal transduction.

In certain aspects the SEMA4D antagonist antibody or fragment thereof for use in the provided method competitively inhibits a reference antibody that includes a variable heavy chain region (VH) including the amino acid sequence SEQ ID NO: 1 and a variable light chain region (VL) including the amino acid sequence SEQ ID NO: 5 from binding to SEMA4D. In certain aspects the SEMA4D antagonist antibody or fragment thereof for use in the provided method binds to the same SEMA4D epitope as a reference antibody that includes a VH including the amino acid sequence SEQ ID NO: 1 and a VL including the amino acid sequence SEQ ID NO: 5.

In certain aspects the SEMA4D antagonist antibody for use in the provided method has a VH and a VL, where the VH includes three complementarity determining regions (CDRs) HCDR1, HCDR2, and HCDR3, where the VL has three CDRs LCDR1, LCDR2, and LCDR3, and where the CDRs include the amino acid sequences SEQ ID NO: 2, SEQ ID NO: 3, SEQ ID NO: 4, SEQ ID NO: 6, SEQ ID NO: 7, and SEQ ID NO: 8, respectively except for at least one or two single conservative amino acid substitutions in one or more of the CDRs. In certain aspects the SEMA4D antagonist antibody for use in the provided method has a VH and a VL, where the VH includes three complementarity determining regions (CDRs) HCDR1, HCDR2, and HCDR3, where the VL includes three CDRs LCDR1, LCDR2, and LCDR3, and where the CDRs include the amino acid sequences SEQ ID NO: 2, SEQ ID NO: 3, SEQ ID NO: 4, SEQ ID NO: 6, SEQ ID NO: 7, and SEQ ID NO: 8, respectively. In certain aspects the VH has an amino acid sequence at least 90% identical to SEQ ID NO: 1 and the VL has an amino acid sequence at least 90% identical to SEQ ID NO: 5. In certain aspects the VH includes the amino acid sequence SEQ ID NO: 1 and the VL includes the amino acid sequence SEQ ID NO: 5.

In certain aspects of the provided method, a first dose of the SEMA4D antagonist antibody is administered, and then the SEMA4D antagonist antibody is administered at least once every week, at least once every two weeks, at least once every three weeks, at least once a month, or at least once every two months thereafter.

In certain aspects of the provided method, the baseline measurement of glucose uptake is measured just prior to the first dose of the SEMA4D antagonist antibody. In certain aspects of the provided method, the change in glucose uptake relative to baseline is measured at least one week after the first dose, at least two weeks after the first dose, at least one month after the first dose, at least two months after the first dose, at least three months after the first dose, at least four months after the first dose, at least five months after the first dose, at least six months after the first dose, or any combination thereof.

In certain aspects of the provided method, glucose uptake in the subject's brain is measured by ¹⁸F-Fluorodeoxyglucose Positron Emission Tomography (FDG-PET) imaging.

In certain aspects of the provided method, the subject is a human. In certain aspects of the provided method, the neurodegenerative or neuroinflammatory disease, disorder or injury is Alzheimer's disease, Parkinson's disease, Huntington's disease, Down syndrome, ataxia, amyotrophic lateral sclerosis (ALS), multiple sclerosis, (MS), epilepsy, meningitis, brain edema, spinal cord injury, traumatic brain injury, frontotemporal dementia (FTD), HIV-related cognitive impairment, CNS Lupus, mild cognitive impairment, or a combination thereof.

In certain aspects of the provided method, the neurodegenerative or neuroinflammatory disease, disorder or injury is Huntington's disease (HD). In certain aspects the subject is at risk of developing HD due to familial history of HD or genetic testing, for example, where genetic testing reveals 36 or more CAG repeats in the subject's HTT gene. In certain aspects the subject is suspected of having HD due to mild motor dysfunction, mild cognitive impairment, or mild neuropsychiatric features. In certain aspects the subject is diagnosed as having HD due to brain atrophy, an elevated Uniform Huntington's Disease Rating Scale score (UHDRS), an increased Huntington's Disease Cognitive Assessment Battery (HD-CAB) score, an increased Huntington's Disease Quantitative Motor Assessment score or a combination thereof. In certain aspects the subject is in the presymptomatic, early prodromal, late prodromal, early manifest, moderate manifest, or advanced manifest stage of HD.

### BRIEF DESCRIPTION OF THE DRAWINGS/FIGURES

FIG. 1 is a schematic of the treatment plan for Cohort A of the SIGNAL clinical study.

FIG. 2 is a schematic and timeline showing the various “groups” of patients to be compared in Cohort A of the SIGNAL clinical study: VV(7-0) is the group treated with VX15 during the first six-month blinded portion of the study; PV(7-0) is the group treated with placebo during the first six-month blinded portion of the study; VV(12-7) is the group treated with VX15 from the beginning of the study, evaluated for the period starting with month 7 through month 11; PV(12-7) is the group that received placebo for the first 6 months of the study and then crossed over at the beginning of month 7 to receive VX15, evaluated for the period starting with month 7 through month 11; VV(12-0) is the group treated with VX15 for the entire study, evaluated from day 0 through month 11; and PV(12-0) is the group that received placebo for the first 6 months of the study and then crossed over at the beginning of month 7 to receive VX15, evaluated for the period starting with day 0 through month 11.

FIG. 3A-B MRI Volume: VV(7-0)-PV(7-0): FIG. 3A shows the difference between the VX15-treated group after 6 months (VV(7-0), n=17) and placebo-treated group after 6 months (PV(7-0), n=19) in least square (LS) mean change in MRI volume expressed in mm³ for each brain region of interest (ROI) during 6 months of treatment with separate measurements for left hemisphere, right hemisphere and the average of left and right hemisphere for that brain region. The bars that bracket each point are the 95% confidence interval. FIG. 3B shows the change in MRI volume for the same groups and brain regions of interest as FIG. 3A, expressed as a % of baseline at start of treatment for each group.

FIG. 4A-B, MRI Volume: PV(12-7)-PV(7-0): FIG. 4A shows the difference between the group treated with placebo for the first 6 months and then treated with VX15 for months 7 to 11, evaluated for the period starting with month 7 through month 11 (PV(12-7), n=19) and the same group when treated with placebo for the first 6 months, evaluated from day zero through the end of month 6 (PV(7-0), n=19), in least square (LS) mean change in MRI volume expressed in mm³ for each brain region of interest (ROI) during 6 months of treatment with separate measurements for left hemisphere, right hemisphere and the average of left and right hemisphere. The bars that bracket each point are the 95% confidence interval. FIG. 4B shows the change in MRI volume for the same groups and brain regions of interest as FIG. 4A, expressed as a % of baseline at start of evaluation for each group.

FIG. 5A-B, MRI Volume: VV(12-7)-PV(7-0): FIG. 5A shows the difference between the VX15-treated group, evaluated for the period starting with month 7 through month 11 (VV(12-7), n=17), and placebo-treated group after 6 months, evaluated from day zero through the end of month 6 (PV(7-0), n=19), in least square (LS) mean change in MRI volume expressed in mm³ for each brain region of interest (ROI). The bars that bracket each point are the 95% confidence interval. FIG. 5B shows the change in MRI volume for the same groups and brain regions of interest as FIG. 5A, expressed as a % of baseline at start of evaluation for each group.

FIG. 6A-B, MRI Volume: VV(12-0)-PV(12-0): FIG. 6A shows the difference between the VX15-treated group, evaluated for the period starting with day 0 through month 11 (VV(12-0), n=17) and placebo/crossover-treated group, evaluated for the period starting with day 0 through month 11 (PV(12-0), n=19), in least square (LS) mean change in MRI volume expressed in mm³ for each brain region of interest (ROI) for the full 11 months of treatment. The bars that bracket each point are the 95% confidence interval. FIG. 6B shows the change in MRI volume for the same groups and brain regions of interest as FIG. 6A, expressed as a % of baseline at start of treatment for each group.

FIG. 7 is a schematic of the link between glutamate uptake and metabolism and glucose transport and glycolysis in astrocytes. Transport of glutamine to neurons for synthesis of glutamate completes the cycle.

FIG. 8A-B, FDG-PET signal change between VV(7-0)-PV(7-0): FIG. 8A shows the difference between the VX15-treated group after 6 months (VV(7-0), n=1) and placebo-treated group after 6 months (PV(7-0), n=8) in least square (LS) mean change in FDG-PET signal expressed in SUV (Standard Uptake Values) for each brain region of interest (ROI) during 6 months of treatment. ROI were defined by co-registration of MRI for that subject and FDG-PET signal was calibrated relative to a reference region (brain stem). For cortical ROI, separate measurements were made for left hemisphere, right hemisphere and the average of left and right hemisphere. The bars that bracket each point are the 95% confidence interval. FIG. 8B shows the change in FDG-PET signal for the same groups and brain regions of interest as FIG. 8A, expressed as a % of baseline at start of treatment for each group.

FIG. 9A-B, FDG-PET signal change between PV(12-7)-PV(7-0): FIG. 9A shows the difference between the group treated with placebo for the first 6 months and then treated with VX15 for months 7 to 11, evaluated for the period starting with month 7 through month 11 (PV(12-7), n=8) and the same group when treated with placebo for the first 6 months, evaluated from day zero through the end of month 6 (PV(7-0), n=8), in least square (LS) mean change in FDG-PET signal for each brain region of interest (ROI). The bars that bracket each point are the 95% confidence interval. FIG. 9B shows the change in FDG-PET signal for the same groups and brain regions of interest as FIG. 9A, expressed as a % of baseline at start of evaluation for each group.

FIG. 10A-B, FDG-PET signal change between VV(12-7)-PV(7-0): FIG. 10A shows the difference between the VX15-treated group, evaluated for the period starting with month 7 through month 11 (VV(12-7), n=11), and placebo-treated group after 6 months, evaluated from day zero through the end of month 6 (PV(7-0), n=8), in least square (LS) mean change in FDG-PET signal for each brain region of interest (ROI). The bars that bracket each point are the 95% confidence interval. FIG. 10B shows the change in FDG-PET signal for the same groups and brain regions of interest as FIG. 10A, expressed as a % of baseline at start of evaluation for each group.

FIG. 11A-B, FDG-PET signal change between VV(12-0)-PV(12-0): FIG. 12A shows the difference between the VX15-treated group, evaluated for the period starting with day 0 through month 11 (VV(12-0), n=11) and placebo/crossover-treated group, evaluated for the period starting with day 0 through month 11 (PV(12-0), n=8), in least square (LS) mean change in FDG-PET signal for each brain region of interest (ROI) for the full 11 months of treatment. The bars that bracket each point are the 95% confidence interval. FIG. 12B shows the change in FDG-PET signal for the same groups and brain regions of interest as FIG. 12A, expressed as a % of baseline at start of treatment for each group.

FIG. 12 shows a schematic of the different VX15 treatment effects observed by MRI and by FDG-PET.

### DETAILED DESCRIPTION

### Definitions

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are also provided.

Amino acids are referred to herein by their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology, but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

The term “polynucleotide” is intended to encompass a singular nucleic acid as well as plural nucleic acids, and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit f-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse ß-glucuronidase.

Disclosed herein are certain binding molecules, or antigen-binding fragments, variants, or derivatives thereof. Unless specifically referring to full-sized antibodies, the term “binding molecule” encompasses full-sized antibodies as well as antigen-binding subunits, fragments, variants, analogs, or derivatives of such antibodies, e.g., engineered antibody molecules or fragments that bind antigen in a manner similar to antibody molecules, but which use a different scaffold.

As used herein, the term “binding molecule” refers in its broadest sense to a molecule that specifically binds to a receptor, e.g., an epitope or an antigenic determinant. As described further herein, a binding molecule can comprise one of more “antigen binding domains” described herein. A non-limiting example of a binding molecule is an antibody or fragment, variant, or derivative thereof that retains antigen-specific binding.

As used herein, the terms “binding domain” or “antigen binding domain” refer to a region of a binding molecule that is necessary and sufficient to specifically bind to an epitope. For example, an “Fv,” e.g., a variable heavy chain and variable light chain of an antibody, either as two separate polypeptide subunits or as a single chain, is considered to be a “binding domain.” Other binding domains include, without limitation, the variable heavy chain (VHH) of an antibody derived from a camelid species, or six immunoglobulin complementarity determining regions (CDRs) expressed in a fibronectin scaffold. A “binding molecule” as described herein can include one, two, three, four, five, six, seven, eight, nine, ten, eleven, twelve or more “antigen binding domains.”

The terms “antibody” and “immunoglobulin” can be used interchangeably herein. An antibody (or a fragment, variant, or derivative thereof as disclosed herein) includes at least the variable domain of a heavy chain (for camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and optionally includes a J chain and/or a secretory component, or an IgM antibody that includes ten or twelve complete heavy chains and ten or twelve complete light chains and optionally includes a J chain.

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, IgA₂, etc. are well characterized and are known to confer functional specialization. Modified versions of each of these classes and isotypes are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure.

Both the light and heavy chains are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant domains of the light chain (CL) and the heavy chain (CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 (or CH4 in the case of IgM) and CL domains actually comprise the carboxy-terminus of the heavy and light chain, respectively.

As indicated above, a variable region (i.e., the “binding domain”) allows the binding molecule to selectively recognize and specifically bind epitopes on antigens. That is, the VL domain and VH domain, or subset of the complementarity determining regions (CDRs), of a binding molecule, e.g., an antibody combine to form the variable region that defines a three dimensional antigen binding site. More specifically, the antigen binding site is defined by three CDRs on each of the VH and VL chains. Certain antibodies form larger structures. For example, IgA can form a molecule that includes two H2L2 units, a J chain, and a secretory component, all covalently connected via disulfide bonds, and IgM can form a pentameric or hexameric molecule that includes five or six H2L2 units and optionally a J chain covalently connected via disulfide bonds.

The six “complementarity determining regions” or “CDRs” present in an antibody antigen-binding domain are short, non-contiguous sequences of amino acids that are specifically positioned to form the binding domain as the antibody assumes its three dimensional configuration in an aqueous environment. The remainder of the amino acids in the binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is generally meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹, or 10⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹, or 10⁷ M⁻¹ sec⁻¹.

A binding molecule, e.g., an antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope by at least 90%, at least 80%, at least 70%, at least 60%, or at least 50%.

As used herein, the term “affinity” refers to a measure of the strength of the binding of an individual epitope with one or more binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of binding domains and an antigen. See, e.g., Harlow at pages 29-34. Avidity is related to both the affinity of individual binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antibody fragments including single-chain antibodies or other binding domains can exist alone or in combination with one or more of the following: hinge region, CH1, CH2, CH3, or CH4 domains, J chain, or secretory component. Also included are antigen-binding fragments that can include any combination of variable region(s) with one or more of a hinge region, CH1, CH2, CH3, or CH4 domains, a J chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al.

As used herein, the term “heavy chain subunit” includes amino acid sequences derived from an immunoglobulin heavy chain, a binding molecule, e.g., an antibody comprising a heavy chain subunit includes at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody or fragment, variant, or derivative thereof can include, in addition to a VH domain, a CH1 domain; CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain aspects a binding molecule, e.g., an antibody or fragment, variant, or derivative thereof can include, in addition to a VH domain, a CH3 domain and a CH4 domain; or a CH3 domain, a CH4 domain, and a J chain. Further, a binding molecule for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule.

The heavy chain subunits of a binding molecule, e.g., an antibody or fragment, variant, or derivative thereof, can include domains derived from different immunoglobulin molecules. For example, a heavy chain subunit of a polypeptide can include a CH1 domain derived from an IgG1 molecule and a hinge region derived from an IgG3 molecule. In another example, a heavy chain subunit can include a hinge region derived, in part, from an IgG1 molecule and, in part, from an IgG3 molecule. In another example, a heavy chain subunit can comprise a chimeric hinge derived, in part, from an IgG1 molecule and, in part, from an IgG4 molecule.

As used herein, the term “light chain subunit” includes amino acid sequences derived from an immunoglobulin light chain. The light chain subunit includes at least one of a VL or CL (e.g., Cκ or Cλ) domain.

Binding molecules, e.g., antibodies or antigen-binding fragments, variants, or derivatives thereof can be described or specified in terms of the epitope(s) or portion(s) of an antigen that they recognize or specifically bind. The portion of a target antigen that specifically interacts with the antigen binding domain of an antibody is an “epitope,” or an “antigenic determinant.” A target antigen can comprise a single epitope or at least two epitopes, and can include any number of epitopes, depending on the size, conformation, and type of antigen.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of a typical immunoglobulin heavy chain molecule.

As used herein the term “CH2 domain” includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat E A et al. op. cit. The CH3 domain extends from the CH2 domain to the C-terminal of the IgG molecule and comprises approximately 108 amino acids. Certain immunoglobulin classes, e.g., IgM, further include a CH4 region.

As used herein, the term “hinge region” includes the portion of a heavy chain molecule that joins the CH1 domain to the CH2 domain. This hinge region comprises approximately 25 amino acids and is flexible, thus allowing the two N-terminal antigen binding regions to move independently.

As used herein the term “disulfide bond” includes the covalent bond formed between two sulfur atoms. The amino acid cysteine comprises a thiol group that can form a disulfide bond or bridge with a second thiol group. In certain IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody, or “bispecific antibody” refer to an antibody that has binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Strohlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which the variable domain in either the heavy and light chain or both is altered by at least partial replacement of one or more amino acids in either the CDR or framework regions. In certain aspects entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain aspects not all of the CDRs are replaced with the complete CDRs from the donor variable region and yet the antigen binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly a portion of a polypeptide that is “carboxy-terminal” or “C-terminal” to another portion of a polypeptide is that portion that comes later in the sequential polypeptide chain. For example in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the term “neuroinflammation” refers to inflammation occurring in the central nervous system (CNS), and is typified by activation of astrocytes and microglial cells, generation of reactive oxygen species, and the expression, e.g., overexpression of proinflammatory cytokines in the brain. Typical proinflammatory cytokines include, but are not limited to interleukin-6 (IL-6), interleukin-1 beta (IL-1β) and tumor necrosis factor alpha (TNF-α). Neuroinflammation can be caused by a disease, disorder, or injury such as multiple sclerosis (MS), meningitis, brain edema, spinal cord injury, traumatic brain injury, viral or bacterial infection, environmental exposure such as pollution or toxins, aging, or a combination thereof. In some instances neuroinflammation can be the cause of subsequent neurodegeneration to the brain, in other instances neuroinflammation can be the result of neurodegeneration.

As used herein, the term “neurodegeneration” refers to the actual breakdown, damage, or death of cells in the CNS and brain, e.g., the loss of structure and/or function of neurons or other brain cells. Neurodegeneration can be the result of neuroinflammation, and/or the cause of neuroinflammation.

As used herein the term “neurodegenerative or neuroinflammatory disease, disorder, or injury” refers to the full scope of diseases, disorders, or injuries that ultimately result in either neuroinflammation or neurodegeneration. Examples include, without limitation, Alzheimer's disease, Parkinson's disease, Huntington's disease, Down syndrome, ataxia, amyotrophic lateral sclerosis (ALS), multiple sclerosis, (MS), epilepsy, meningitis, brain edema, spinal cord injury, traumatic brain injury, frontotemporal dementia (FTD), HIV-related cognitive impairment, CNS Lupus, mild cognitive impairment, or a combination thereof.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder. Terms such as “prevent,” “prevention,” “avoid,” “deterrence” and the like refer to prophylactic or preventative measures that prevent the development of an undiagnosed targeted pathologic condition or disorder. Thus, “those in need of treatment” can include those already with the disorder; those prone to have the disorder; and those in whom the disorder is to be prevented.

The term “therapeutically effective amount” refers to an amount of an antibody, polypeptide, polynucleotide, small organic molecule, or other drug effective to “treat” a disease, disorder, or injury in a subject or mammal. In the case of cancer, the therapeutically effective amount of the drug can reduce the number of cancer cells; retard or stop cancer cell division, reduce or retard an increase in tumor size; inhibit, e.g., suppress, retard, prevent, stop, delay, or reverse cancer cell infiltration into peripheral organs including, for example, the spread of cancer into soft tissue and bone; inhibit, e.g., suppress, retard, prevent, shrink, stop, delay, or reverse tumor metastasis; inhibit, e.g., suppress, retard, prevent, stop, delay, or reverse tumor growth; relieve to some extent one or more of the symptoms associated with the cancer, reduce morbidity and mortality; improve quality of life; or a combination of such effects. To the extent the drug prevents growth and/or kills existing cancer cells, it can be referred to as cytostatic and/or cytotoxic.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” includes subjects, such as mammalian subjects, that would benefit from administration of a therapy as described herein.

As used herein, the term “healthcare provider” refers to individuals or institutions that directly interact and/or administer therapies to living subjects, e.g., human patients. Non-limiting examples of healthcare providers include doctors, nurses, technicians, therapists, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for obtaining data and/or the examination, and/or the processing of data obtained from a living subject and/or materials derived from a living subject, e.g., a human being. Non-limiting examples of processing include radiographic (e.g., X-rays), fluorographic, tomographic (e.g., Positron Emission Tomography or PET-scans), or magnetic resonance (MRI) imaging of a subject, biological, biochemical, serological, chemical, immunohematological, hematological, biophysical, cytological, pathological, genetic, or other examination of materials derived from the human body, for the purpose of providing data or information, e.g., for the diagnosis, prevention, or treatment of any disease or impairment of, or the assessment of the health of living subjects, e.g., human beings. These examinations can include procedures obtain imaging data of the subject, to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a therapy to treat a particular disease, disorder, or injury. A healthcare provider can implement or instruct another healthcare provider or patient, both under the first healthcare provider's control, to perform the following actions: submit to an imaging study or perform an imaging study on a patient, obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy (e.g., comparing baseline imaging results with results obtained following a treatment regimen), commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, imaging studies, collection of a sample, processing of a sample, submission of a sample, receipt of a sample, transfer of a sample, analysis or measurement a sample, quantification a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent. In certain aspects, a healthcare benefits provider can authorize or deny treatment based on the results of a companion diagnostic assay, e.g., imaging studies that show whether a certain therapy is effective in a given individual patient.

In some aspects, a clinical laboratory can, for example, perform imaging studies on a patient under orders from a healthcare provider, compare baseline and follow-on imaging studies after a given therapy is administered, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities. A clinical laboratory typically performs tests ordered by a healthcare provider or a healthcare benefits provider, and typically works under the healthcare provider's and/or healthcare benefits provider's control, or in a joint enterprise with healthcare provider and/or healthcare benefits provider.

### Target Polypeptide Description—SEMA4D

As used herein, the terms “semaphorin-4D”, “SEMA4D”, and “SEMA4D polypeptide” are used interchangeably, as are “SEMA4D” and “Sema4D.” In certain embodiments, SEMA4D is expressed on the surface of or secreted by a cell. In another embodiment, SEMA4D is membrane bound. In another embodiment, SEMA4D is soluble, e.g., sSEMA4D. In another embodiment, SEMA4D can include a full-sized SEMA4D or a fragment thereof, or a SEMA4D variant polypeptide, where the fragment of SEMA4D or SEMA4D variant polypeptide retains some or all functional properties of the full-sized SEMA4D.

The full-sized human SEMA4D protein is a homodimeric transmembrane protein consisting of two polypeptide chains of 150 kDa. SEMA4D belongs to the semaphorin family of cell surface receptors and is also referred to as CD100. Both human and mouse SEMA4D/Sema4D are proteolytically cleaved from their transmembrane form to generate 120-kDa soluble forms, giving rise to two Sema4D isoforms (Kumanogoh et al., J. Cell Science 116(7):3464 (2003)). Semaphorins consist of soluble and membrane-bound proteins that were originally defined as axonal-guidance factors which play an important role in establishing precise connections between neurons and their appropriate target. Structurally considered a class IV semaphorin, SEMA4D consists of an amino-terminal signal sequence followed by a characteristic ‘Sema’ domain, which contains 17 conserved cysteine residues, an Ig-like domain, a lysine-rich stretch, a hydrophobic transmembrane region, and a cytoplasmic tail.

The SEMA4D polypeptide includes a signal sequence of about 13 amino acids followed by a semaphorin domain of about 512 amino acids, an immunoglobulin-like (Ig-like) domain of about 65 amino acids, a lysine-rich stretch of 104 amino acids, a hydrophobic transmembrane region of about 19 amino acids, and a cytoplasmic tail of 110 amino acids. A consensus site for tyrosine phosphorylation in the cytoplasmic tail supports the predicted association of SEMA4D with a tyrosine kinase (Schlossman et al., Eds. (1995) Leucocyte Typing V (Oxford University Press, Oxford).

SEMA4D is known to have at least three functional receptors, Plexin-B1, Plexin-B2 and CD72. Plexin-B1, is expressed in non-lymphoid tissues and has been shown to be a high affinity (1 nM) receptor for SEMA4D (Tamagnone et al., Cell 99:71-80 (1999)). SEMA4D stimulation of Plexin B1 signaling has been shown to induce growth cone collapse of neurons, and to induce process extension collapse and apoptosis of oligodendrocytes (Giraudon et al., J. Immunol. 172:1246-1255 (2004); Giraudon et al., NeuroMolecular Med. 7:207-216 (2005)). After binding to SEMA4D, Plexin B1 signaling mediates the inactivation of R-Ras, leading to a decrease in the integrin mediated attachment to the extracellular matrix, as well as to activation of RhoA, leading to cell collapse by reorganization of the cytoskeleton. See Kruger et al., Nature Rev. Mol. Cell Biol. 6:789-800 (2005); Pasterkamp, TRENDS in Cell Biology 15:61-64 (2005)). Plexin-B2 has an intermediate affinity for SEMA4D and a recent report indicates that PLXNB2 is expressed on keratinocytes and activates SEMA4D-positive γδ T cells to contribute to epithelial repair (Witherden et al., Immunity. 2012 Aug. 24; 37(2):314-25).

In lymphoid tissues, CD72 is utilized as a low affinity (300 nM) SEMA4D receptor (Kumanogoh et al., Immunity 13:621-631 (2000)). B cells and Antigen Presenting Cells (APC) express CD72, and anti-CD72 antibodies have many of the same effects as sSEMA4D, such as enhancement of CD40-induced B cell responses and B cell shedding of CD23. CD72 is thought to act as a negative regulator of B cell responses by recruiting the tyrosine phosphatase SHP-1, which can associate with many inhibitory receptors. Interaction of SEMA4D with CD72 results in the dissociation of SHP-1, and the loss of this negative activation signal. SEMA4D has been shown to promote T cell stimulation and B cell aggregation and survival in vitro. The addition of SEMA4D-expressing cells or sSEMA4D enhances CD40-induced B cell proliferation and immunoglobulin production in vitro, and accelerates in vivo antibody responses (Ishida et al., Inter. Immunol. 15:1027-1034 (2003); Kumanogoh and H. Kukutani, Trends in Immunol. 22:670-676 (2001)). sSEMA4D enhances the CD40 induced maturation of DCs, including up-regulation of costimulatory molecules and increased secretion of IL-12. In addition, sSEMA4D can inhibit immune cell migration, which can be reversed by addition of blocking anti-SEMA4D mouse antibodies (Elhabazi et al., J. Immunol. 166:4341-4347 (2001); Delaire et al., J. Immunol. 166:4348-4354 (2001)).

Sema4D is expressed at high levels in lymphoid organs, including the spleen, thymus, and lymph nodes, and in non-lymphoid organs, such as the brain, heart, and kidney. In lymphoid organs, Sema4D is abundantly expressed on resting T cells but only weakly expressed on resting B cells and antigen-presenting cells (APCs), such as dendritic cells (DCs).

Cellular activation increases the surface expression of SEMA4D as well as the generation of soluble SEMA4D (sSEMA4D). The expression pattern of SEMA4D suggests that it plays an important physiological as well as pathological role in the immune system. SEMA4D has been shown to promote B cell activation, aggregation and survival; enhance CD40-induced proliferation and antibody production; enhance antibody response to T cell dependent antigens; increase T cell proliferation; enhance dendritic cell maturation and ability to stimulate T cells; and is directly implicated in demyelination and axonal degeneration (Shi et al., Immunity 13:633-642 (2000); Kumanogoh et al., J Immunol 169:1175-1181 (2002); and Watanabe et al., J Immunol 167:4321-4328 (2001)).

### Anti-SEMA4D Antibodies

Antibodies that bind SEMA4D have been described in the art. See, for example, US Publ. Nos. 2008/0219971 A1, US 2010/0285036 A1, and US 2006/0233793 A1, International Patent Applications WO 93/14125, WO 2008/100995, and WO 2010/129917, and Herold et al., Int. Immunol. 7(1): 1-8 (1995), each of which is herein incorporated in its entirety by reference. In certain aspects antibodies provided herein are SEMA4D antagonist antibodies, in that they interfere with, inhibit, block, or destroy one or more activities or functions of SEMA4D

In certain embodiments, the SEMA4D antagonist antibody blocks the interaction of SEMA4D with one or more of its receptors, e.g., Plexin-B1, Plexin-B2, and/or CD72. Anti-SEMA4D antibodies having these properties can be used in the methods provided herein. Antibodies that can be used include, but are not limited to MAbs VX15/2503, 67, 76, 2282 and antigen-binding fragments, variants, or derivatives thereof which are fully described in US 2010/0285036 A1 and US 2008/0219971 A1. Mab VX15/2503 is also referred to herein as “VX15,” and the terms can be used interchangeably. VX15 comprises a heavy chain variable region with the amino acid sequence SEQ ID NO: 1 and a light chain variable region with the amino acid sequence SEQ ID NO: 5. Additional antibodies which can be used in the methods provided herein include the BD16 antibody described in US 2006/0233793 A1 as well as antigen-binding fragments, variants, or derivatives thereof; or any of MAb 301, MAb 1893, MAb 657, MAb 1807, MAb 1656, MAb 1808, Mab 59, MAb 2191, MAb 2274, MAb 2275, MAb 2276, MAb 2277, MAb 2278, MAb 2279, MAb 2280, MAb 2281, MAb 2282, MAb 2283, MAb 2284, and MAb 2285, as well as any fragments, variants or derivatives thereof as described in US 2008/0219971 A1. In certain embodiments an anti-SEMA4D antibody for use in the methods provided herein binds human, murine, or both human and murine SEMA4D. Also useful are antibodies which bind to the same epitope as any of the aforementioned antibodies and/or antibodies which competitively inhibit binding or activity of any of the aforementioned antibodies.

In certain embodiments, an anti-SEMA4D antibody or antigen-binding fragment, variant, or derivative thereof useful in the methods provided herein has an amino acid sequence that has at least about 80%, about 85%, about 88%, about 89%, about 90%, about 91%, about 92%, about 93%, about 94%, or about 95% sequence identity to the amino acid sequence for a reference anti-SEMA4D antibody molecule, for example, those described above. In a further embodiment, the binding molecule shares at least about 96%, about 97%, about 98%, about 99%, or 100% sequence identity to a reference antibody.

In certain aspects, the SEMA4D antagonist antibody or antigen-binding fragment, variant, or derivative thereof can inhibit SEMA4D interaction with its receptor, e.g., Plexin-B1, Plexin-B2, or CD72. In certain aspects the SEMA4D antagonist antibody or antigen-binding fragment, variant, or derivative thereof can inhibit SEMA4D-mediated Plexin-B1 signal transduction.

In certain aspects, the SEMA4D antagonist antibody or antigen-binding fragment, variant, or derivative thereof competitively inhibits a reference antibody comprising a variable heavy chain region (VH) comprising the amino acid sequence SEQ ID NO: 1 and a variable light chain region (VL) comprising the amino acid sequence SEQ ID NO: 5 from binding to SEMA4D. In certain aspects, the SEMA4D antagonist antibody or antigen-binding fragment, variant, or derivative thereof binds to the same SEMA4D epitope as a reference antibody comprising a VH comprising the amino acid sequence SEQ ID NO: 1 and a VL comprising the amino acid sequence SEQ ID NO: 5. In certain aspects, the VH of the SEMA4D antagonist antibody or antigen-binding fragment, variant, or derivative thereof comprises three complementarity determining regions (CDRs) HCDR1, HCDR2, and HCDR3, and the VL comprises three cCDRs LCDR1, LCDR2, and LCDR3, the CDRs comprising the amino acid sequences SEQ ID NO: 2, SEQ ID NO: 3, SEQ ID NO: 4, SEQ ID NO: 6, SEQ ID NO: 7, and SEQ ID NO: 8, respectively except for at least one, two, three, four, five, or six single conservative amino acid substitutions in one or more of the CDRs. In certain aspects the CDRs comprise the amino acid sequences SEQ ID NO: 2, SEQ ID NO: 3, SEQ ID NO: 4, SEQ ID NO: 6, SEQ ID NO: 7, and SEQ ID NO: 8, respectively.

In certain aspects the VH of the SEMA4D antagonist antibody or antigen-binding fragment, variant, or derivative thereof comprises an amino acid sequence at least 70%, 75%, 80%, 85%, 90%, 95%, or 100% identical to SEQ ID NO: 1 and the VL of the SEMA4D antagonist antibody or antigen-binding fragment, variant, or derivative thereof comprises an amino acid sequence at least 70%, 75%, 80%, 85%, 90%, 95%, or 100% identical to SEQ ID NO: 5; or the VH comprises an amino acid sequence at least 70%, 75%, 80%, 85%, 90%, 95%, or 100% identical to SEQ ID NO: 9 and the VL comprises an amino acid sequence at least 70%, 75%, 80%, 85%, 90%, 95%, or 100% identical to SEQ ID NO: 10. In certain aspects, the VH comprises the amino acid sequence SEQ ID NO: 1 and the VL comprises the amino acid sequence SEQ ID NO: 5; or the VH comprises the amino acid sequence SEQ ID NO: 9 and the VL comprises the amino acid sequence SEQ ID NO: 10.

Also included for use in the methods provided herein are polypeptides encoding anti-SEMA4D antibodies, or antigen-binding fragments, variants, or derivatives thereof as described herein, polynucleotides encoding such polypeptides, vectors comprising such polynucleotides, and host cells comprising such vectors or polynucleotides, all for producing anti-SEMA4D antibodies, or antigen-binding fragments, variants, or derivatives thereof for use in the methods described herein.

Suitable biologically active variants of the SEMA4D antagonist antibodies of the disclosure can be used in the methods of the present disclosure. Such variants will retain the desired binding properties of the parent anti-SEMA4D antibody. Methods for making antibody variants are generally available in the art.

Methods for mutagenesis and nucleotide sequence alterations are well known in the art. See, for example, Walker and Gaastra, eds. (1983) Techniques in Molecular Biology (MacMillan Publishing Company, New York); Kunkel, Proc. Natl. Acad. Sci. USA 82:488-492 (1985); Kunkel et al., Methods Enzymol. 154:367-382 (1987); Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest can be found in the model of Dayhoff et al. (1978) in Atlas of Protein Sequence and Structure (Natl. Biomed. Res. Found., Washington, D.C.), pp. 345-352, herein incorporated by reference in its entirety. The model of Dayhoff et al. uses the Point Accepted Mutation (PAM) amino acid similarity matrix (PAM 250 matrix) to determine suitable conservative amino acid substitutions. In certain aspects, conservative substitutions, such as exchanging one amino acid with another having similar properties are used. Examples of conservative amino acid substitutions as taught by the PAM 250 matrix of the Dayhoff et al. model include, but are not limited to, Gly↔Ala, Val↔Ile↔Leu, Asp↔Glu, Lys↔Arg, Asn↔Gln, and Phe↔Trp↔Tyr.

In constructing variants of the SEMA4D antagonist binding molecule, e.g., an antibody or antigen-binding fragment thereof, polypeptides of interest, modifications are made such that variants continue to possess the desired properties, e.g., being capable of specifically binding to a SEMA4D, e.g., human, murine, or both human and murine SEMA4D, e.g., expressed on the surface of or secreted by a cell and having SEMA4D blocking activity, as described herein. In certain aspects, mutations made in the DNA encoding the variant polypeptide maintain the reading frame and do not create complementary regions that could produce secondary mRNA structure. See EP Patent Application Publication No. 75,444.

Methods for measuring anti-SEMA4D binding molecule, e.g., an antibody or antigen-binding fragment, variant, or derivative thereof, binding specificity include, but are not limited to, standard competitive binding assays, assays for monitoring immunoglobulin secretion by T cells or B cells, T cell proliferation assays, apoptosis assays, ELISA assays, and the like. See, for example, such assays disclosed in WO 93/14125; Shi et al., Immunity 13:633-642 (2000); Kumanogoh et al., J Immunol 169:1175-1181 (2002); Watanabe et al., JImmunol 167:4321-4328 (2001); Wang et al., Blood 97:3498-3504 (2001); and Giraudon et al., J Immunol 172(2):1246-1255 (2004), all of which are herein incorporated by reference.

Methods for measuring the anti-angiogenic ability of an anti-SEMA4D antibody or antigen-binding fragment, variant, or derivative thereof are well known in the art.

When discussed herein whether any particular polypeptide, including the constant regions, CDRs, VH domains, or VL domains disclosed herein, is at least about 65%, about 70%, about 75%, about 80%, about 85%, about 90%, about 91%, about 92%, about 93%, about 94%, about 95%, about 96%, about 97%, about 98%, about 99%, or even about 100% identical to another polypeptide, the % identity can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman (1981) Adv. Appl. Math. 2:482-489, to find the best segment of homology between two sequences. When using BESTFIT or any other sequence alignment program to determine whether a particular sequence is, for example, 95% identical to a reference sequence according to the present disclosure, the parameters are set, of course, such that the percentage of identity is calculated over the full length of the reference polypeptide sequence and that gaps in homology of up to 5% of the total number of amino acids in the reference sequence are allowed.

For purposes of the present disclosure, percent sequence identity can be determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant can, for example, differ from a reference anti-SEMA4D antibody (e.g., MAb VX15/2503, 67, 76, or 2282) by as few as 1 to 15 amino acid residues, as few as 1 to 10 amino acid residues, such as 6-10, as few as 5, as few as 4, 3, 2, or even 1 amino acid residue.

The constant region of an anti-SEMA4D antibody can be mutated to alter effector function in a number of ways. For example, see U.S. Pat. No. 6,737,056B1 and U.S. Patent Application Publication No. 2004/0132101A1, which disclose Fc mutations that optimize antibody binding to Fc receptors.

In certain anti-SEMA4D antibodies or fragments, variants or derivatives thereof useful in the methods provided herein, the Fc portion can be mutated to decrease effector function using techniques known in the art. For example, the deletion or inactivation (through point mutations or other means) of a constant region domain can reduce Fc receptor binding of the circulating modified antibody thereby increasing tumor localization. In other cases, constant region modifications consistent with the instant disclosure moderate complement binding and thus reduce the serum half-life. Yet other modifications of the constant region can be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, can easily be measured and quantified using well known immunological techniques without undue experimentation.

Anti-SEMA4D antibodies for use in the methods provided herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, etc. Additionally, the derivative can contain one or more non-classical amino acids.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an anti-SEMA4D polypeptide, or to block SEMA4D interaction with its receptor).

For example, it is possible to introduce mutations only in framework regions or only in CDR regions of an antibody molecule. Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to immunospecifically bind at least one epitope of a SEMA4D polypeptide) can be determined using techniques described herein or by routinely modifying techniques known in the art.

In certain embodiments, the SEMA4D antagonist antibodies for use in the methods provided herein comprise at least one optimized complementarity-determining region (CDR). By “optimized CDR” is intended that the CDR has been modified and optimized to improve binding affinity and/or anti-SEMA4D activity that is imparted to an anti-SEMA4D antibody comprising the optimized CDR. “Anti-SEMA4D activity” or “SEMA4D blocking activity” can include activity which modulates one or more of the following activities associated with SEMA4D: B cell activation, aggregation and survival; CD40-induced proliferation and antibody production; antibody response to T cell dependent antigens; T cell or other immune cell proliferation; dendritic cell maturation; demyelination and axonal degeneration; apoptosis of pluripotent neural precursors and/or oligodendrocytes; induction of endothelial cell migration; inhibition of spontaneous monocyte migration; inhibition, delay, or reduction of tumor cell growth or metastasis, binding to cell surface plexin B1 or other receptor, or any other activity association with soluble SEMA4D or SEMA4D that is expressed on the surface of SEMA4D+ cells. In a particular embodiment, anti-SEMA4D activity includes the ability to inhibit, delay, or reduce tumor metastases, either in combination with inhibition, delay, or reduction of primary tumor cell growth and tumor metastases, or independently of primary tumor cell growth and tumor metastases. Anti-SEMA4D activity can also be attributed to a decrease in incidence or severity of diseases associated with SEMA4D expression, including, but not limited to, certain types of cancers including lymphomas, autoimmune diseases, inflammatory diseases including central nervous system (CNS) and peripheral nervous system (PNS) inflammatory diseases, transplant rejections, and invasive angiogenesis. Examples of optimized antibodies based on murine anti-SEMA4D MAb BD16 were described in US Publ. No. 2008/0219971 A1, International Patent Application WO 93/14125 and Herold et al., Int. Immunol. 7(1): 1-8 (1995), each of which are herein incorporated by reference in their entirety. The modifications can involve replacement of amino acid residues within the CDR such that an anti-SEMA4D antibody retains specificity for the SEMA4D antigen and has improved binding affinity and/or improved anti-SEMA4D activity.

### Astrocytes and Astrocyte Activation

Astrocytes are specialized glial cells that perform many essential complex functions in the healthy CNS, including regulation of blood flow, fluid/ion/pH/neurotransmitter homeostasis, synapse formation/function, energy and metabolism, and blood-brain barrier maintenance (Barres B A, Neuron 60:430-440 (2008). Importantly, astrocytes respond to CNS injury through a process referred to as reactive astrogliosis, resulting in “reactive astrocytes,” or “astrocyte activation,” the terms used interchangeably herein. Reactive astrogliosis can serve as a major pathological hallmark of neuroinflammatory and neurodegenerative diseases. Increasing evidence points towards the potential of reactive astrogliosis to play either primary or contributing roles in CNS disorders via loss of normal astrocyte functions or gain of abnormal activities. Given their central role in many CNS diseases, there is a significant need to identify and rigorously test new molecular targets that restore normal astrocyte function to effectively slow or even reverse disease progression. There are several potential pathways through which astrocytes can impact CNS diseases.

Astrocytes can play a central role in brain function by affecting the activity of neurons through the distribution of energy substrates from the circulation (e.g., glucose uptake from capillaries) to neurons. See P. Lecca, Technical Report CoSBi July 2007, University of Trento Centre for Computational and Systems Biology, available at www.cosbi.eu/research/publications?pdf=5041 (last visited Jan. 30, 2017). Lecca reports that neurons contribute at most 50% of cerebral cortical volume and that the astrocytes outnumber the neurons (Id.). A single astrocyte can make contact with many cells. Astrocytes are star-shaped cells with multiple fine projections, which cover the entire surface of the capillaries that feed the brain. Thus, astrocytes form the first cellular barrier encountered by glucose entering the brain parenchyma. Therefore astrocytes are a major site of glucose uptake in the CNS (Id.).

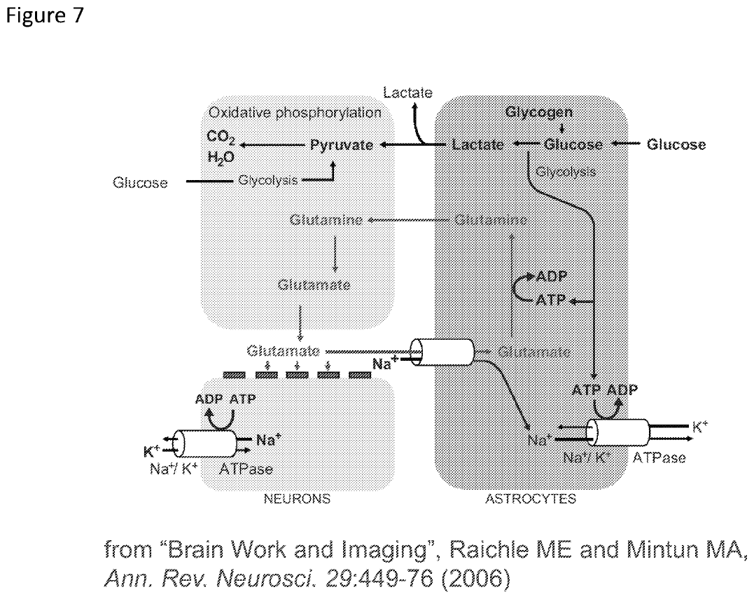

The uptake of glucose in astrocyte is triggered by glutamate (see FIG. 7, from Raichle M E and Mintun M A, Ann. Rev. Neurosci. 29:449-76 (2006)). Raichle and Mintun notes that glutamate uptake triggers nonoxidative glucose utilization in astrocytes (aerobic glycolysis) and glucose uptake from the circulation through the glucose transporter GLUT 1. Glutamate is the main excitatory neurotransmitter of the cerebral cortex (FIG. 7). See also, Hertz, L et al., J Cerebr. Blood Flow Metab. 27:219-249 (2007), and Kasiscke, H D, et al., Science 305:99-103 (2004). Astrocyte projections cradle the synapses between neurons and can take up free glutamate and convert it to glutamine (Maragakis, N J and J D Rothstein Nature Clinical Practice/Neurology 2:679-689 (2006)). The uptake and conversion takes energy, requiring two ATP molecules per glutamate taken up. Regulation of glutamate levels at synapses is important because excess excitatory transmitter can trigger excitotoxicity and neurodegeneration.

During reactive astrogliosis, however, astrocytes can pull back their projections, and no longer cradle the synapses or take up excess glutamate. Accordingly, glucose metabolism in astrocytes, and in particular uptake of glucose from capillaries, can be reduced. Reactive astrocytes downregulate the glutamate receptor and associated glycolysis and glucose transport, which can be detected as a reduced FDG-PET signal. Reactive astrogliosis is a major pathological hallmark of neuroinflammatory and neurodegenerative diseases. Increasing evidence points towards the potential of reactive astrogliosis to play either primary or contributing roles in CNS disorders via loss of normal astrocyte functions or gain of abnormal activities. Given their central role in many CNS diseases, there is a significant need to identify and rigorously test new molecular targets that restore normal astrocyte function to effectively slow or even reverse disease progression. There are several potential pathways through which astrocytes can impact CNS diseases.

Astrocytes and oligodendrocyte precursor cell (OPC) support. Demyelination that occurs in neuroinflammatory diseases, such as Multiple Sclerosis, is associated with marked destruction and loss of cells comprising the oligodendrocyte lineage (Ozawa K, et al. Brain 117:1311-1322 (1994)). Endogenous remyelination mechanisms fail during the recovery phase in part because of the inability of OPCs to fully differentiate into mature myelinating oligodendrocytes (Wolswijk G. Brain 123:105-115 (2000)). Data obtained from other experimentally induced demyelination models indicate that newly maturing OPCs, in contrast to surviving mature oligodendrocytes, are required for remyelination during the recovery phase (Levine J M, Reynolds R. Exp Neurol. 160:333-347 (1999)). Astrocytes have been shown to play a significant role in supporting the function and viability of the oligodendrocyte lineage. For example, Talbott and colleagues showed that in ethidium bromide-induced demyelinated lesions, astrocytes are required for Nkx2.2+/Olig2+ OPCs to fully differentiate into oligodendrocytes and carry out remyelination (Talbott, J F, et al., Exp Neurol. 192:11-24 (2005)). Arai and Lo demonstrated in vitro that astrocytes provide soluble trophic factor support to OPCs that protect these cells from increased oxidative stress (Arai, K. and Lo, E. H. J. Neurosci. Res. 88: 758-763 (2010)). Others have shown that inhibition of astrocyte activation in the settings of experimental autoimmune encephalomyelitis, experimental optic neuritis, and spinal cord injury leads to improved remyelination profiles and functional outcome measures (Brambilla R, et al., J Immunol 182:2628-2640 (2009); Brambilla R, et al., J Neuroinflammation 9:213; Brambilla R, et al, J Exp Med 202:145-156 (2005)).

Given the role that astrocytes play in facilitation of OPC survival and function, the juxtaposition of SEMA4D-expressing OPCs and SEMA4D receptor-expressing astrocytes suggests that disease-related activation of astrocytes with associated upregulation of plexin-B receptors and SEMA4D signaling can affect OPC function.

Astrocytes and Neuronal Support.

Accumulating evidence indicates that astrocytes play roles in synaptic transmission through the regulated release of synaptically active molecules including glutamate, purines (ATP and adenosine), GABA, and D-serine (reviewed by Halassa M M et al., Trends Mol Med 13:54-63 (2007); Nedergaard M et al. Trends Neurosci 26:523-530 (2003)). The release of such gliotransmitters occurs in response to changes in neuronal synaptic activity, involves astrocyte excitability as reflected by increases in astrocyte calcium signaling, and can alter neuronal excitability (Id.). In addition to having direct effects on synaptic activity via the release of gliotransmitters, astrocytes have the potential to exert powerful and long-term influences on synaptic function through the release of growth factors and related molecules (Barres B A Neuron 60:430-440 (2008)).

Astrocytes and blood brain barrier (BBB) integrity. Astrocytes play an essential role in formation of the blood-brain barrier (BBB) and in regulating transport across the BBB, a homeostatic process critical for proper neuronal function. The BBB is a highly complex brain endothelial structure of the differentiated neurovascular system comprised of pericytes, astrocytes, and endothelial cells. BBB compromise has been implicated in a number of neurodegenerative diseases, including meningitis, brain edema, epilepsy, Alzheimer's disease (AD), Parkinson's disease (PD), stroke, amyotrophic lateral sclerosis (ALS), and Multiple Sclerosis (MS); reviewed by (Zlokovic B V Nat Rev Neurosci. 12:723-738 (2011)).

Astrocytes are “polarized” cells in that they extend specialized membranous processes comprised of unique cellular machinery and membrane components that interact with specific cell types. For example, astrocytic processes proximal to cerebral microvessels or pia are characterized by a high density of the water channel, aquaporin 4 (Aqp4) (Neely J D, et al., Proc Natl Acad Sci USA 98:14108-14113 (2001); Amiry-Moghaddam M, et al., Proc Natl Acad Sci USA 100:2106-2111 (2003)). In contrast, astrocytic processes facing synaptic regions are enriched in glutamate transporters, while the density of Aqp4 is comparatively low (Nielsen S et al., (1997) J Neurosci 17:171-180 (1997); Chaudhry F A et al., Neuron 15:711-720 (1995)). Astrocytic polarization is disrupted in a brain undergoing neurodegeneration. For example, in the setting of Alzheimer's disease, Aqp4 staining intensities significantly decrease in regions with significant amyloid plaque burden. In fact, Yang and colleagues showed that the accumulation of amyloid pathology in tg-ArcSwe AD mice is coupled temporally and spatially to loss of astrocyte polarization (Yang J L, et al., J Alzheimer's Dis. 27:711-22 (2011)).

Role of SEMA4D Signaling in Promoting Astrocyte Activation and Reactive Astrogliosis.

Given the association of SEMA4D receptor expression and the astrocyte activation marker GFAP, there exists the possibility that SEMA4D signaling can potentiate astrocyte activation, thereby providing a “feed-forward” mechanism during disease states. See, e.g., U.S. Pat. No. 9,249,227, incorporated herein by reference in its entirety.

Increased Glucose Uptake in Brain Regions as an Early Indicator of SEMA4D Antagonist Antibody Treatment Efficacy

The disclosure provides an early biomarker test to determine whether SEMA4D antagonist antibody treatment is likely to be effective in treating a neuroinflammatory or neurodegenerative disease, disorder, or injury in a subject. The test entails measuring a baseline level of brain glucose uptake in the patient, e.g., by ¹⁸F-Fluorodeoxyglucose Positron Emission Tomography (FDG-PET) imaging, administering one or more initial doses of the SEMA4D antagonist antibody to the subject, and then remeasuring glucose uptake in the subjects brain. Patients who are suffering from a an accumulated deficit, sometimes referred to herein as an historical deficit, in brain glucose uptake, e.g., due to accumulation of reactive astrocytes in the disease pathology as explained elsewhere herein, will present as responsive to therapy with the SEMA4D antagonist antibody when the remeasurement of glucose uptake, e.g., via FDG-PET, shows an increase as compared to the baseline measurement. This would indicate that the deficit in glucose uptake is related to a pathogenic mechanism that can be reversed by SEMA4D antagonist. The deficit in glucose uptake could have developed over a period of weeks, months or years prior to initiation of treatment. If no increase in the FDG-PET signal is observed, a conclusion can be made that the patient is not responsive to the SEMA4D antagonist antibody therapy either because the patient has no deficit in brain glucose uptake, or the pathogenic basis of such deficit in that disease or that patient cannot be reversed by SEMA4D antagonist therapy. Either way, treatment with the SEMA4D antagonist antibody could then be adjusted or discontinued.

In certain aspects, the disclosure provides a method for determining whether a semaphorin 4D (SEMA4D) antagonist antibody or antigen-binding fragment, variant, or derivative thereof will be effective in treating a defined or specific neurodegenerative or neuroinflammatory disease, disorder, or injury, where the method includes: administering an effective amount of a SEMA4D antagonist antibody or antigen-binding fragment, variant, or derivative thereof to a subject having, suspected of having, or at risk of developing that neurodegenerative or neuroinflammatory disease, disorder, or injury; measuring the level of glucose uptake in the subject's brain relative to a baseline level of glucose uptake in the subject's brain measured prior to administration of the SEMA4D antagonist antibody or fragment, variant, or derivative thereof; and continuing administration of the SEMA4D antagonist antibody or fragment, variant, or derivative thereof if an increase in glucose uptake over baseline is detected; or discontinuing or adjusting administration of the SEMA4D antagonist antibody or fragment, variant, or derivative thereof if no change or a decrease in glucose uptake relative to baseline is detected.

In certain aspects, the brain glucose uptake measurements can be carried out, e.g., by a clinical laboratory, under a healthcare provider's clear direction and control. In certain aspects, a healthcare provider can order the brain glucose uptake measurements to be performed. In certain aspects the brain glucose uptake measurements can be performed by a clinical laboratory, and the clinical laboratory can then instruct or advise the healthcare provider of the best treatment for the subject or patient. For example, the method can include measuring, e.g., by a clinical laboratory, the baseline level of glucose uptake in the brain of a subject presented as having, suspected of having, or at risk of developing a neurodegenerative or neuroinflammatory disease, disorder, or injury; and then remeasuring the level of glucose uptake in the subject's brain following administration of a SEMA4D antagonist antibody or antigen-binding fragment, variant, or derivative thereof to the subject by a healthcare provider; and then instructing the healthcare provider to continue administration of the SEMA4D antagonist antibody or fragment, variant, or derivative thereof if an increase in brain glucose uptake over baseline is detected; or instructing the healthcare provider to discontinue administration of the SEMA4D antagonist antibody or fragment, variant, or derivative thereof if no change or a decrease in glucose uptake relative to baseline is detected. In certain aspects administration of the SEMA4D antagonist antibody and measurement of glucose uptake in the subject's brain can be carried out by the same person or facility. In certain aspects, the methods as provided herein can be ordered by a healthcare benefits provider prior to authorizing payment for further treatment with the SEMA4D antagonist antibody.

As will be understood by those of ordinary skill in the art, an “effective dose” of a SEMA4D antagonist antibody can vary between individual subjects or patients. The disclosure further provides a method in which to determine an effective does for an individual subject. By measuring changes in glucose uptake in an individual subject's brain, a healthcare provider can use the method provided herein to adjust dosing to find the most effective dose for a given subject. For example, if no change or only a small change is seen in glucose uptake over baseline after a given administration of a SEMA4D antagonist antibody, the dosage of the antibody can be increased followed by a remeasurement of glucose uptake in the subject's brain. If a change is then seen, the healthcare provider can continue treatment of the subject with that dosage. In certain aspects, multiple measurements of glucose uptake can be taken to “fine-tune” the optimal SEMA4D antagonist antibody dosage for a given subject or patient. Care must, however, be taken to allow sufficient time for an historical or contemporary deficit to accumulate that might be subject to reversal by treatment with SEMA4D antagonist. This can be established for each disease of interest by delaying resumption of treatment from several months to, in a very slowly progressing disease, several years.

The disclosure further provides a method for treating a subject having, suspected of having, or at risk of developing a neurodegenerative or neuroinflammatory disease, disorder, or injury, where the method includes: administering a SEMA4D antagonist antibody or antigen-binding fragment, variant, or derivative thereof to a subject having, suspected of having, or at risk of developing a neurodegenerative or neuroinflammatory disease, disorder, or injury; measuring the level of glucose uptake in the subject's brain relative to a baseline level of glucose uptake in the subject's brain measured prior to administration, e.g., by FDG-PET; and continuing administration of the SEMA4D antagonist antibody or fragment, variant, or derivative thereof if an increase in glucose uptake over baseline is detected; or discontinuing or adjusting administration of the SEMA4D antagonist antibody or fragment, variant, or derivative thereof if no change or a decrease in glucose uptake relative to baseline is detected. The treatment method can further include measuring the baseline level of glucose uptake in the brain of the subject, or in some aspects such baseline measurement can have been performed previously.

As noted above, the treatment method can further be “fine-tuned” by additional measurements of glucose uptake in the subject's brain following adjustments in dosing of the SEMA4D antagonist antibody. Moreover, the glucose uptake measurements of the treatment can be carried out, e.g., by a clinical laboratory, under a healthcare provider's clear direction and control. In certain aspects, a healthcare provider can order the glucose uptake measurements to be performed as part of a treatment regimen. In certain aspects the glucose uptake measurements can be performed by a clinical laboratory, and the clinical laboratory can then instruct or advise the healthcare provider regarding treatment of the subject or patient. In certain aspects administration of the SEMA4D antagonist antibody and measurement of glucose uptake in the subject's brain can be carried out by the same person or facility. In certain aspects, the methods as provided herein can be ordered by a healthcare benefits provider prior to authorizing payment for further treatment with the SEMA4D antagonist antibody.

In certain aspects, the SEMA4D antagonist antibody or fragment, variant, or derivative thereof for use in the methods provided herein can inhibit SEMA4D interaction with its receptor, e.g., Plexin-B1, Plexin-B2, or CD72. In certain aspects, the SEMA4D antagonist antibody or fragment, variant, or derivative thereof can inhibit SEMA4D-mediated Plexin-B1 signal transduction. In certain aspects the SEMA4D antagonist antibody or fragment, variant, or derivative thereof is related to the SEMA4D antagonist antibody VX15. For example in certain aspects the SEMA4D antagonist antibody or fragment, variant, or derivative thereof can competitively inhibit or bind to the same epitope as VX15, i.e., a reference antibody comprising a variable heavy chain region (VH) comprising the amino acid sequence SEQ ID NO: 1 and a variable light chain region (VL) comprising the amino acid sequence SEQ ID NO: 5 from binding to SEMA4D. In certain aspects the SEMA4D antagonist antibody has a VH with three complementarity determining regions (CDRs) HCDR1, HCDR2, and HCDR3 and a VL with three CDRs LCDR1, LCDR2, and LCDR3, where the CDRs comprise the amino acid sequences SEQ ID NO: 2, SEQ ID NO: 3, SEQ ID NO: 4, SEQ ID NO: 6, SEQ ID NO: 7, and SEQ ID NO: 8, respectively except for at least one, two, three, four, five, or six single conservative amino acid substitutions in one or more of the CDRs. In certain aspects the SEMA4D antagonist antibody has a VH with three complementarity determining regions (CDRs) HCDR1, HCDR2, and HCDR3, and a VL with three CDRs LCDR1, LCDR2, and LCDR3, and where the CDRs comprise the amino acid sequences SEQ ID NO: 2, SEQ ID NO: 3, SEQ ID NO: 4, SEQ ID NO: 6, SEQ ID NO: 7, and SEQ ID NO: 8, respectively. In certain aspects the VH of the SEMA4D antagonist antibody has an amino acid sequence at least 70%, at least 75%, at least 80%, at least 85%, at least 90%, at least 95% or 100% identical to SEQ ID NO: 1 and the VL of the SEMA4D antagonist antibody has an amino acid sequence at least 70%, at least 75%, at least 80%, at least 85%, at least 90%, at least 95% or 100% identical to SEQ ID NO: 5; or the VH has an amino acid sequence at least 70%, at least 75%, at least 80%, at least 85%, at least 90%, at least 95% or 100% identical to SEQ ID NO: 9 and the VL has an amino acid sequence at least 70%, at least 75%, at least 80%, at least 85%, at least 90%, at least 95% or 100% identical to SEQ ID NO: 10. In certain aspects the VH of the SEMA4D antagonist antibody has the amino acid sequence SEQ ID NO: 1 and the VL of the SEMA4D antagonist antibody has the amino acid sequence SEQ ID NO: 5; or the VH of the SEMA4D antagonist antibody has the amino acid sequence SEQ ID NO: 9 and the VL of the SEMA4D antagonist antibody has the amino acid sequence SEQ ID NO: 10.

According to the methods provided herein, a first dose of the SEMA4D antagonist antibody can be administered, and then additional doses of the SEMA4D antagonist antibody can be administered thereafter, e.g., at least once every week, at least once every two weeks, at least once every three weeks, at least once a month, or at least once every two months, and so on. If the treatment is found to be effective according to the methods provided herein, administration of the SEMA4D antibody can be continued for as long as needed, in some cases, throughout the lifetime of the subject or patient or in other cases for a discrete period of time until the subject's or patient's neuroinflammatory or neurodegenerative disease, disorder, or injury is under control, is cured, or the symptoms abate.

According to the methods provided herein, the baseline measurement of glucose uptake in the subject's brain is typically measured just prior to the first dose of the SEMA4D antagonist antibody, but can, in certain instances take place earlier, or in certain instances can take place immediately after the first dose of the SEMA4D antagonist antibody. In those methods in which adjustments to dosages are being assessed, a new “baseline” measurement can be taken some time after the first dose, from several months to several years depending on the rate of disease progression. Relative to the baseline measurement, recovery of the accumulated or “historical” deficit in glucose uptake that is a marker of effective treatment with a SEMA4D antagonist antibody can occur rapidly following the first dose of the SEMA4D antagonist antibody, or may take a period of time, or multiple doses of the SEMA4D antagonist antibody to be observable using available technology, e.g., FDG-PET. Accordingly, remeasurement of glucose uptake in the subject's brain relative to baseline can occur, e.g., at least one week after the first dose, at least two weeks after the first dose, at least one month after the first dose, at least two months after the first dose, at least three months after the first dose, at least four months after the first dose, at least five months after the first dose, at least six months after the first dose, or even later, or any combination thereof.

In certain aspects, the patient or subject undergoing the methods as provided herein is a mammalian subject, e.g., a rodent, a non-human primate, or a human subject.

The neurodegenerative or neuroinflammatory disease, disorder or injury that can benefit from administration of a SEMA4D antagonist antibody according to the methods provided herein can be, e.g., Alzheimer's disease, Parkinson's disease, Huntington's disease, Down syndrome, ataxia, amyotrophic lateral sclerosis (ALS), multiple sclerosis, (MS), epilepsy, meningitis, brain edema, spinal cord injury, traumatic brain injury, frontotemporal dementia (FTD), HIV-related cognitive impairment, CNS Lupus, mild cognitive impairment, or a combination thereof.

In certain aspects the disease is Huntington's disease (HD), as described elsewhere herein. In certain aspects the subject is at risk of developing HD due to familial history of HD or genetic testing, e.g., a test showing that the subject's HTT gene comprises 36 or more CAG repeats. A benefit of the methods as provided herein is that such subjects often exhibit no outward symptoms of HD, but are clearly at risk. As shown in the Examples, early treatment can provide a benefit over later treatment, and such individuals can be evaluated as to whether they could benefit from treatment with a SEMA4D antagonist antibody according to the methods provided herein long before they exhibit any outward symptoms. In certain aspects the subject is suspected of having HD due to, e.g., mild motor dysfunction, mild cognitive impairment, or mild neuropsychiatric features. Tests for determining such mild dysfunctions are well known to those of ordinary skill in the art and are available in the literature, e.g., in Bates, G P, et al., Nature Reviews/Disease Primers 1:1-21 (2015). In certain aspects, the subject is already diagnosed as having HD due to, e.g., an elevated Uniform Huntington's Disease Rating Scale score (UHDRS), an increased Huntington's Disease Cognitive Assessment Battery (HD-CAB) score, quantitative motor assessments or a combination thereof. In those subjects known to be at risk of HD, the subject can be in the presymptomatic stage of HD, the early prodromal stage of HD, late prodromal stage of HD, and, following diagnosis, the early manifest stage of HD, the moderate manifest stage of HD, or advanced manifest stage of HD. The signs and symptoms of the various stages of HD can be found, e.g., in Bates, G P, et al., Nature Reviews/Disease Primers 1:1-21 (2015).

### Treatment Methods Using SEMA4D Antagonist Antibodies

The diagnostic methods of the disclosure relate to the use of SEMA4D antagonist antibodies, including antigen-binding fragments, variants, and derivatives thereof, to treat a subject having a neuroinflammatory or neurodegenerative disease, disorder, or injury, or to evaluate whether the subject will benefit from treatment with a SEMA4D antagonist antibody. Though the following discussion refers to administration of a SEMA4D antagonist antibody, the methods described herein are also applicable to the antigen-binding fragments, variants, and derivatives of a SEMA4D antagonist antibody or other biologics or small molecules that retain the desired properties of a SEMA4D antagonist antibody of the disclosure, e.g., capable of specifically binding SEMA4D, e.g., human, mouse, or human and mouse SEMA4D, having SEMA4D neutralizing activity, and/or blocking the interaction of SEMA4D with its receptor, e.g., Plexin-B1.

In one aspect, the disclosure provides the administration of a SEMA4D antagonist antibody or antigen binding fragment thereof or other biologic or small molecule that binds and neutralizes SEMA4D as described herein to a patient, where the patient has, is suspected of having, or has the risk of developing a neuroinflammatory or neurodegenerative disease, disorder, or injury. In another aspect, treatment is also intended to include the administration of a pharmaceutical composition comprising the SEMA4D antagonist antibody or antigen binding fragment thereof to a patient, where the patient has, is suspected of having, or has the risk of developing a neuroinflammatory or neurodegenerative disease, disorder, or injury.

The SEMA4D antagonist antibodies or binding fragments thereof as described herein are useful for the treatment of various neuroinflammatory or neurodegenerative diseases, disorders, or injuries. In some aspects, treatment is intended to induce an improvement in the symptoms associated with the disease, disorder, or injury. In other embodiments, treatment is intended to reduce, retard or stop an increase in symptom manifestations. In other aspects, treatment is intended to inhibit, e.g., suppress, retard, prevent, stop, or reverse a manifestation of symptoms. In other aspects, treatment is intended to relieve to some extent one or more of the symptoms associated with the disorder. In these situations, the symptoms can be, e.g., neuropsychiatric symptoms, cognitive symptoms, and/or motor dysfunction. In other aspects, treatment is intended to reduce morbidity and mortality. In other aspects, treatment is intended to improve quality of life.

In one aspect, the disclosure relates to the use of SEMA4D antagonist antibodies or antigen-binding fragments, variants, or derivatives thereof, as a medicament, in particular for use in the treatment of various neuroinflammatory or neurodegenerative diseases, disorders, or injuries to improve the symptoms associated with the disorder.

In accordance with the methods of the present disclosure, at least one SEMA4D antagonist antibody or antigen binding fragment, variant, or derivative thereof, or other biologic or small molecule as defined elsewhere herein can be used to promote a positive therapeutic response with respect to the neuroinflammatory or neurodegenerative disease, disorder, or injury. A “positive therapeutic response” with respect to the neuroinflammatory or neurodegenerative disease, disorder, or injury is intended to include an improvement in the symptoms associated with the disorder. Such positive therapeutic responses are not limited to the route of administration and can comprise administration to the donor, the donor tissue (such as for example organ perfusion), the host, any combination thereof, and the like. In particular, the methods provided herein are directed to inhibiting, preventing, reducing, alleviating, or lessening the progression of a neuroinflammatory or neurodegenerative disease, disorder, or injury in a patient. Thus, for example, an improvement in the disorder can be characterized as an absence of clinically observable symptoms, a decrease in the incidence of clinically observable symptoms, or a change in the clinically observable symptoms.

The SEMA4D antagonist antibodies or antigen binding fragments, variants, or derivatives thereof or other biologics or small molecules can be used in combination with at least one or more other treatments for neuroinflammatory or neurodegenerative diseases, disorders, or injuries; where the additional therapy is administered prior to, during, or subsequent to the SEMA4D antagonist antibody or antigen binding fragment, variant, or derivative thereof, therapy. Thus, where the combined therapies comprise administration of a SEMA4D antagonist antibody or antigen binding fragment, variant, or derivative thereof, in combination with administration of another therapeutic agent, the methods of the disclosure encompass coadministration, using separate formulations or a single pharmaceutical formulation, with simultaneous or consecutive administration in either order.

In certain aspects the neuroinflammatory or neurodegenerative disease, disorder, or injury can be, e.g., Alzheimer's disease, Parkinson's disease, Huntington's disease, Down syndrome, ataxia, amyotrophic lateral sclerosis (ALS), frontotemporal dementia (FTD), HIV-related cognitive impairment, CNS Lupus, mild cognitive impairment, multiple sclerosis, epilepsy, meningitis, or a combination thereof. In certain aspects of any of the aforementioned procedures, the neurodegenerative disease is Huntington's disease,

In some aspects, a healthcare provider can administer or instruct another healthcare provider to administer a therapy comprising an effective amount of a SEMA4D antagonist antibody, where the subject has, is suspected to have, or is at risk of contracting a neuroinflammatory or neurodegenerative disease, disorder, or injury. A healthcare provider can implement or instruct another healthcare provider or patient to perform the following actions: obtain a sample or image, process a sample or image, submit a sample or image, receive a sample or image, transfer a sample or image, analyze or measure a sample or image, quantify a sample or image, provide the results obtained after analyzing/measuring/quantifying a sample or image, receive the results obtained after analyzing/measuring/quantifying a sample or image, compare/score the results obtained after analyzing/measuring/quantifying one or more samples or images, provide the comparison/score from one or more samples, obtain the comparison/score from one or more samples or images, administer a therapy, e.g., an effective amount of a SEMA4D antagonist antibody, commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, performing imaging, collection of a sample or image, processing of a sample or image, submission of a sample or image, receipt of a sample or image, transfer of a sample or image, analysis or measurement a sample or image, quantification a sample or image, provision of results obtained after analyzing/measuring/quantifying a sample or image, transfer of results obtained after analyzing/measuring/quantifying a sample or image, comparison/scoring of results obtained after analyzing/measuring/quantifying one or more samples or images, transfer of the comparison/score from one or more samples or images, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition, a healthcare benefits provider can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample or image, process a sample or image, submit a sample or image, receive a sample or image, transfer a sample or image, analyze or measure a sample or image, quantify a sample or image, provide the results obtained after analyzing/measuring/quantifying a sample or image, receive the results obtained after analyzing/measuring/quantifying a sample or image, compare/score the results obtained after analyzing/measuring/quantifying one or more samples or images, provide the comparison/score from one or more samples or images, obtain the comparison/score from one or more samples or images, or other related activities.

In certain aspects, any of the aforementioned procedures can be used to determine if a subject has a neuroinflammatory or neurodegenerative disease, disorder, or injury in which changes in glucose uptake can be a pathogenic factor subject to treatment with a SEMA4D antagonist.

In some aspects, a healthcare provider, clinical laboratory, or other entity can, for example, collect or obtain an image, process an image, submit an image, receive an image, transfer an image, analyze or measure an image, quantify an image, provide the results obtained after analyzing/measuring/quantifying an image, receive the results obtained after analyzing/measuring/quantifying an image, compare/score the results obtained after analyzing/measuring/quantifying one or more images, provide the comparison/score from one or more images, obtain the comparison/score from one or more images, or other related activities. Images that can be used in such aspects include, but are not limited to, images obtained by angiography, ultrasound, computed tomography (CT), magnetic resonance imaging (MRI), positron emission tomography (PET), e.g., FDG-PET, optical coherence tomography (OCT), near-infrared spectroscopy (NIRS), and NIR fluorescence. In certain embodiments, imaging techniques that have been described in the literature can be used (Tardif et al. Circ Cardiovasc Imaging 4:319-333 (2011)).

### Pharmaceutical Compositions and Administration Methods

Methods of preparing and administering SEMA4D antagonist antibodies, or antigen-binding fragments, variants, or derivatives thereof to a subject in need thereof are well known to or are readily determined by those skilled in the art. The route of administration of the SEMA4D antagonist antibody, or antigen-binding fragment, variant, or derivative thereof, can be, for example, oral, parenteral, by inhalation or topical. The term parenteral as used herein includes, e.g., intravenous, intraarterial, intraperitoneal, intramuscular, subcutaneous, rectal, or vaginal administration. While all these forms of administration are clearly contemplated as being within the scope of the disclosure, an example of a form for administration would be a solution for injection, in particular for intravenous or intraarterial injection or drip. A suitable pharmaceutical composition for injection can comprise a buffer (e.g. acetate, phosphate or citrate buffer), a surfactant (e.g. polysorbate), optionally a stabilizer agent (e.g. human albumin), etc. However, in other methods compatible with the teachings herein, SEMA4D antagonist antibodies or antigen-binding fragments, variants, or derivatives thereof can be delivered directly to the site of the adverse cellular population thereby increasing the exposure of the diseased tissue to the therapeutic agent.

As discussed herein, SEMA4D antagonist antibodies or antigen-binding fragments, variants, or derivatives thereof can be administered in a pharmaceutically effective amount for the in vivo treatment of neuroinflammatory or neurodegenerative diseases, disorders, or injuries. In this regard, it will be appreciated that the disclosed SEMA4D antagonist antibodies can be formulated so as to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of a SEMA4D antagonist antibody or antigen-binding fragment, variant, or derivative thereof, shall be held to mean an amount sufficient to achieve effective binding to a target and to achieve a benefit, e.g., improve the symptoms associated with a neurodegenerative disorder. The diagnostic methods provided herein allow the skilled person to determine and/or “fine-tune” the effective amount of a SEMA4D antagonist antibody or fragment thereof for any individual subject or patient.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include, e.g., water, alcoholic/aqueous solutions, emulsions or suspensions, including saline and buffered media. In the subject disclosure, pharmaceutically acceptable carriers include, but are not limited to, 0.01-0.1 M, e.g., about 0.05 M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as, for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and can be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences (Mack Publishing Co.) 16th ed. (1980).

Prevention of the action of microorganisms can be achieved by various antibacterial and antifungal agents, for example, parabens, chlorobutanol, phenol, ascorbic acid, thimerosal and the like. In many cases, isotonic agents can be included, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

In any case, sterile injectable solutions can be prepared by incorporating an active compound (e.g., a SEMA4D antagonist antibody or antigen-binding fragment, variant, or derivative thereof, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation include vacuum drying and freeze-drying, which yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture can have labels or package inserts indicating that the associated compositions are useful for treating a subject suffering from, or predisposed to a disease or disorder.

Parenteral formulations can be a single bolus dose, an infusion or a loading bolus dose followed with a maintenance dose. These compositions can be administered at specific fixed or variable intervals, e.g., once a day, or on an “as needed” basis.

Certain pharmaceutical compositions used in this disclosure can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a SEMA4D antagonist antibody, or fragment, variant, or derivative thereof, to be combined with the carrier materials to produce a single dosage form will vary depending upon the host treated and the particular mode of administration, and can be determined according to the methods provided herein. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, SEMA4D antagonist antibodies, or antigen-binding fragments, variants, or derivatives thereof can be administered to a human or other animal in accordance with the aforementioned methods of treatment in an amount sufficient to produce a therapeutic effect. The SEMA4D antagonist antibodies or antigen-binding fragments, variants or derivatives thereof can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of SEMA4D antagonist antibodies or antigen-binding fragments, variants, or derivatives thereof, of the disclosure can be used.

The amount of a SEMA4D antagonist antibody or binding fragment, variant, or derivative thereof, to be administered is readily determined by one of ordinary skill in the art according to the present disclosure. Factors influencing the mode of administration and the respective amount of a SEMA4D antagonist antibody, antigen-binding fragment, variant or derivative thereof include, but are not limited to, the severity of the disease, the history of the disease, and the age, height, weight, health, and physical condition of the individual undergoing therapy. Similarly, the amount of a SEMA4D antagonist antibody or fragment, variant, or derivative thereof, to be administered will be dependent upon the mode of administration and whether the subject will undergo a single dose or multiple doses of this agent.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevier, Amsterdam); Goldsby et al., eds. (2000) Kuby Immunology (4th ed.; H. Freeman and & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlag); Sambrook and Russell (2001) Molecular Cloning: A Laboratory Manual (Cold Spring Harbor Press); Lewin (2003) Genes VIII (Prentice Hall 2003); Harlow and Lane (1988) Antibodies: A Laboratory Manual (Cold Spring Harbor Press); Dieffenbach and Dveksler (2003) PCR Primer (Cold Spring Harbor Press).

All of the references cited above, as well as all references cited herein, are incorporated herein by reference in their entireties.

The following examples are offered by way of illustration and not by way of limitation.

### EXAMPLES

### Example 1: Clinical Protocol

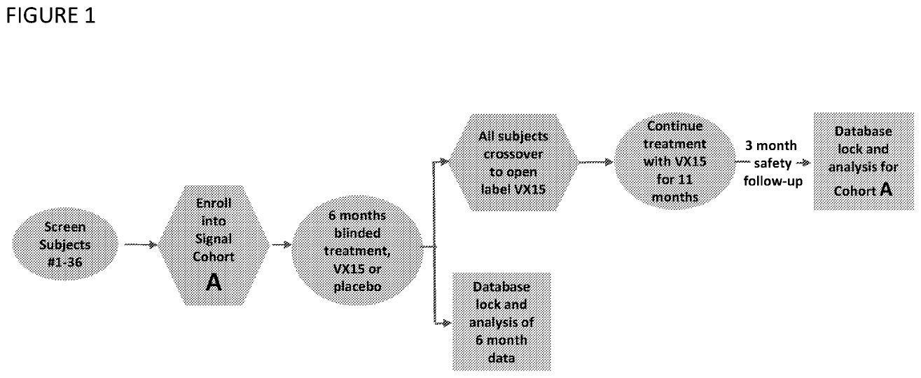

The protocol for Cohort A of the Signal Clinical Trial is shown in FIG. 1. Thirty-six (36) individuals who were 21 years of age or older with late prodromal (CAG-age product score (CAP score) of greater than 200 and Diagnostic Confidence Level (DCL) of 2 or 3) or early manifest HD (Total Functional Capacity (TFC) greater than or equal to 11) were enrolled into Signal Cohort A. All of the enrolled subjects had also undergone genetic testing with a known CAG repeat greater than or equal to 36. All subjects were capable of and provided informed consent for study participation. The subjects were randomized with 17 patients assigned to the VX15 treatment group, and 19 patients assigned to the placebo treatment group. 21 prodromal and 15 early manifest status subjects were enrolled. A Summary of the patients enrolled in the study is shown in Table 2

TABLE 2
Patient Data

| | | | Late | Early |
|---|---|---|---|---|
| | | | Prodromal | Manifest |
| | | All | HD | HD |
| Gender | | | | |
| Female | N | 22 | 15 | 7 |
| | % | 61.1 | 71.4 | 46.7 |
| Male | N | 14 | 6 | 8 |
| | % | 38.9 | 28.6 | 53.3 |
| Hispanic | | | | |
| No | N | 36 | 21 | 15 |
| | % | 100.0 | 100.0 | 100.0 |
| White | | | | |
| Yes | N | 36 | 21 | 15 |
| | % | 100.0 | 100.0 | 100.0 |
| Black or African American | | | | |
| Yes | N | 36 | 21 | 15 |
| | % | 100.0 | 100.0 | 100.0 |
| Asian | | | | |
| No | N | 36 | 21 | 15 |
| | % | 100.0 | 100.0 | 100.0 |
| Native Hawaiian or | | | | |
| Other Pacific Islander | | | | |
| No | N | 36 | 21 | 15 |
| | % | 100.0 | 100.0 | 100.0 |
| American Indian or | | | | |
| Alaska Native | | | | |
| No | N | 36 | 21 | 15 |
| | % | 100.0 | 100.0 | 100.0 |
| Other Race | | | | |
| No | N | 36 | 21 | 15 |
| | % | 100.0 | 100.0 | 100.0 |
| Age | N | 36 | 21 | 15 |
| | Mean | 46.78 | 42.57 | 52.67 |
| | Std | 12.68 | 12.68 | 10.41 |
| | Min | 22.00 | 22.00 | 32.00 |
| | Max | 68.00 | 64.00 | 68.00 |
| Onset Age | N | | | 15 |
| | Mean | | | 49.93 |
| | Std | | | 11.36 |
| | Min | | | 31.54 |
| | Max | | | 63.81 |
| Years Since Onset | N | | | 15 |
| | Mean | | | 3.25 |
| | Std | | | 3.23 |
| | Min | | | 0.05 |
| | Max | | | 9.84 |
| Education Years | N | 36 | 21 | 15 |
| | Mean | 14.44 | 14.05 | 15.00 |
| | Std | 2.21 | 1.80 | 2.65 |
| | Min | 9.00 | 12.00 | 9.00 |
| | Max | 20.00 | 18.00 | 20.00 |
| Montreal Cognitive | N | 36 | 21 | 15 |
| | Mean | 26.61 | 26.95 | 26.13 |
| | Std | 1.82 | 1.80 | 1.81 |
| | Min | 23.00 | 23.00 | 23.00 |
| | Max | 30.00 | 30.00 | 28.00 |
| TFC | N | 36 | 21 | 15 |
| | Mean | 12.03 | 12.29 | 11.67 |
| | Std | 0.88 | 0.85 | 0.82 |
| | Min | 11.00 | 11.00 | 11.00 |
| | Max | 13.00 | 13.00 | 13.00 |
| Functional Assesstment | N | 36 | 21 | 15 |
| | Min | 24.17 | 24.10 | 24.27 |
| | Std | 1.08 | 1.26 | 0.80 |
| | Min | 21.00 | 21.00 | 23.00 |
| | Max | 25.00 | 25.00 | 25.00 |
| Q69: Independenee | N | 36 | 21 | 15 |
| | Mean | 93.89 | 96.19 | 90.67 |
| | Std | 6.67 | 6.10 | 6.23 |
| | Min | 80.00 | 80.00 | 80.00 |
| | Max | 100.00 | 100.00 | 100.00 |
| Total Motor | N | 36 | 21 | 15 |
| | Mean | 14.72 | 10.43 | 20.73 |
| | Std | 8.95 | 4.07 | 10.50 |
| | Min | 4.00 | 4.00 | 10.00 |
| | Max | 47.00 | 20.00 | 47.00 |
| PROBLEM BEHAVIORS | N | 36 | 21 | 15 |
| ASSESSMENT | Mean | 7.50 | 8.76 | 5.73 |
| | Std | 7.73 | 8.81 | 5.71 |
| | Min | 0.00 | 0.00 | 0.00 |
| | Max | 29.00 | 29.00 | 19.00 |
| Diagnostic Confidence Level | | | | |
| abnormalities may be HD | N | 18 | 18 | · |
| signs 50%-89% confident | % | 50.0 | 85.7 | · |
| abnormalities, likely HD | N | 3 | 3 | · |
| signs 90%-98% confident | % | 8.3 | 14.3 | · |
| abnormalities, unequivocal | N | 15 | · | 15 |
| HD signs >99% confident | % | 41.7 | · | 100.0 |
| Allele 1(from Lab) | N | 36 | 21 | 15 |
| | Mean | 42.56 | 43.00 | 41.93 |
| | Std | 2.96 | 3.13 | 2.69 |
| | Min | 38.00 | 38.00 | 38.00 |
| | Max | 52.00 | 52.00 | 49.00 |
| Allele 2(from Lab) | N | 36 | 21 | 15 |
| | Mean | 18.39 | 17.29 | 19.93 |
| | Std | 3.65 | 2.70 | 4.30 |
| | Min | 9.00 | 9.00 | 15.00 |
| | Max | 30.00 | 23.00 | 30.00 |
| CAP (age*(Allele 1-33.66)) | N | 36 | 21 | 15 |
| | Mean | 388.29 | 366.57 | 418.71 |
| | Std | 78.00 | 63.56 | 87.98 |
| | Min | 195.30 | 247.38 | 195.30 |
| | Max | 569.74 | 485.68 | 569.74 |

As shown in FIG. 1, the Cohort A subjects were treated for 6 months with either VX15 or placebo and then all subjects were treated with VX15 for an additional 5 months, followed by 3 months of follow up.

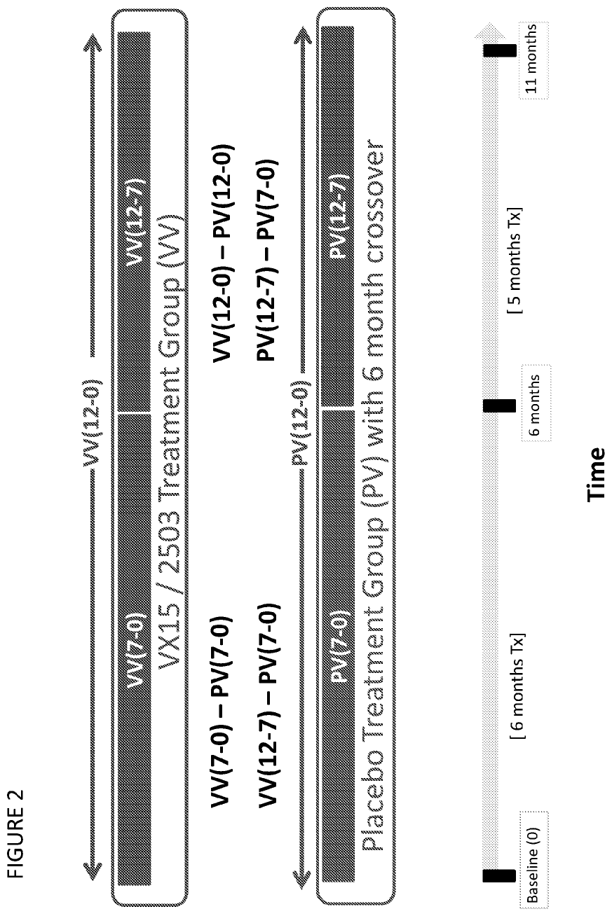

The Cohort A patients were treated with 6 monthly intravenous doses of VX15 (n=17) at 20 mg/kg, or placebo (n=19). This portion of the study was blinded. After the initial 6 months, all subjects enrolled in Cohort A continued the study with open-label treatment with VX15/2503 for 5 additional months. The various study groups and a timeline of the treatments is shown in FIG. 2 which also indicates the nomenclature used below to describe different treatment regimens and time frames, e.g. PV(7-0) indicates the group that is treated with placebo (P) during the first 6 months and with VX15 (V) during the next 5 months and focuses on change in MRI volume or FDG-PET signal between the baseline at study start, visit 0, and visit 7 at the end of 6 months, (7-0). This is the control employed for comparison to all other 5 or 6 month VX15 treatment periods. At each monthly visit, the patients were screened for safety, tolerability, and efficacy. Blood samples were tested for total serum soluble SEMA4D (sSEMA4D). In the course of the monthly visits, efficacy assessments were administered at regular intervals. These included the Huntington's Disease-Cognitive Assessment Battery (HD-CAB) and quantitative motor (Q-Motor) battery. The HD-CAB can differentiate control, pre-HD, and early HD subjects. The battery has high sensitivity to disease status, with large effect sizes, and high reliability, and well-characterized psychometrics and practice effects (Stout J C, et al., Mov Disord. 29:1281-1288 (2014). Motor symptoms in HD can be objectively assessed using Q-Motor assessments. The Q-Motor battery includes assessments of different motor tasks related to functionally relevant everyday tasks (see, e.g., Tabrizi S J, et al., Lancet Neurol. 8:791-801 (2009). At baseline, t=0, and during the monthly visit 7 (v7) at the end of 6 months of treatment and visit 12 (v12) at the end of 11 months of treatment all the patients underwent MRI imaging and a subset of patients received FDG-PET imaging. Multiple brain regions were analyzed for changes in the MRI and FDG-PET signals. The primary brain regions of interest (ROI) for MRI are shown in Tables 3 and 4 and for FDG-PET in Table 5. Where applicable, separate measurements were made for left and right hemispheres and their average was also calculated.

TABLE 3
MRI CORTICAL VOLUME MEASURES

| Precedent | |
|---|---|
| Interest* | ROI |
| Primary | Precentral gyrus |
| Primary | Supramarginal gyrus |
| Primary | Superior temporal gyrus |
| Primary | Middle temporal gyrus |
| Primary | Rostral middle frontal gyrus |
| Secondary | Caudal middle frontal gyms |
| Secondary | Pars opercularis |
| Secondary | Pars triangularis |
| Secondary | Pars Orbitalis |
| Secondary | Inferior temporal gyms |
| Secondary | Transverse temporalcortex |
| Secondary | Superior frontal gyms |
| Secondary | Paracentral lobule |
| Secondary | Post-central gryus |
| Secondary | Precunues cortex |
| Secondary | Lingual gyms |
| Secondary | Pericalcarine cortex |
| Secondary | Cuneus cortex |
| Secondary | Lateral occipital cortex |
| Secondary | Rostral anterior cingulate cortex |
| Secondary | Caudal anterior cingulate cortex |
| Secondary | Posterior cingulate cortex |
| Secondary | Inferior parietal |
| Secondary | Superior parietal |
| Secondary | Medial Orbitofrontal |

TABLE 4
MRI VOLUME MEASURES

| Precedent | |
|---|---|
| Interest* | ROI |
| Primary | Caudate |
| Primary | Putamen |
| Primary | Total white matter |
| Secondary | Hippocampus |
| Secondary | Amygdala |
| Secondary | Globus Pallidus |
| Secondary | Thalamus |

TABLE 5
BRAIN ROI FOR FDG-PET UPTAKE MEASURES

| | |
|---|---|
| Subcortical FDG uptake measures: | Thalamus |
| | Caudate |
| | Putamen |
| | Pallidum |
| | Hippocampus |
| | Amygdala |
| | Ventral diencephalon |
| | Brainstem |
| | Lateral Ventricle |
| | Inferior Lateral Ventricle |
| | 3rd Ventricle |
| | 4th Ventricle |
| | 5th Ventricle |
| Cortical FDG uptake measures: | Entorhinal cortex |
| | Fusiform |
| | Inferior parietal |
| | Inferior temporal |
| | Middle temporal |
| | Parahippocampal |
| | Superior frontal |
| | Superior parietal |
| | Superior temporal |
| | Temporal pole |
| Global FDG uptake measures: | Forebrain parenchyma |
| | Intracranial volume |
| | Cerebellar white matter |
| | Cerebellar gray matter |
| | Cortical white matter |
| | White matter hypointensities |

After all Cohort A subjects received 6 months of blinded treatment, an analysis of primary data of the double-blind portion of Cohort A was completed. After all Cohort A subjects completed 11 months of treatment, an analysis of imaging data was completed.

The treatments were well-tolerated and compliance was excellent. No concerning safety signals were identified.

Statistical Methods Analyses followed the Intention To Treat (ITT) principle and used standard statistical methods, Fisher's exact test, chi-square and logistic regression for categorical data, two-sample t-tests, analysis of covariance and mixed-effect model with repeated measures (MMRM) for continuous data.

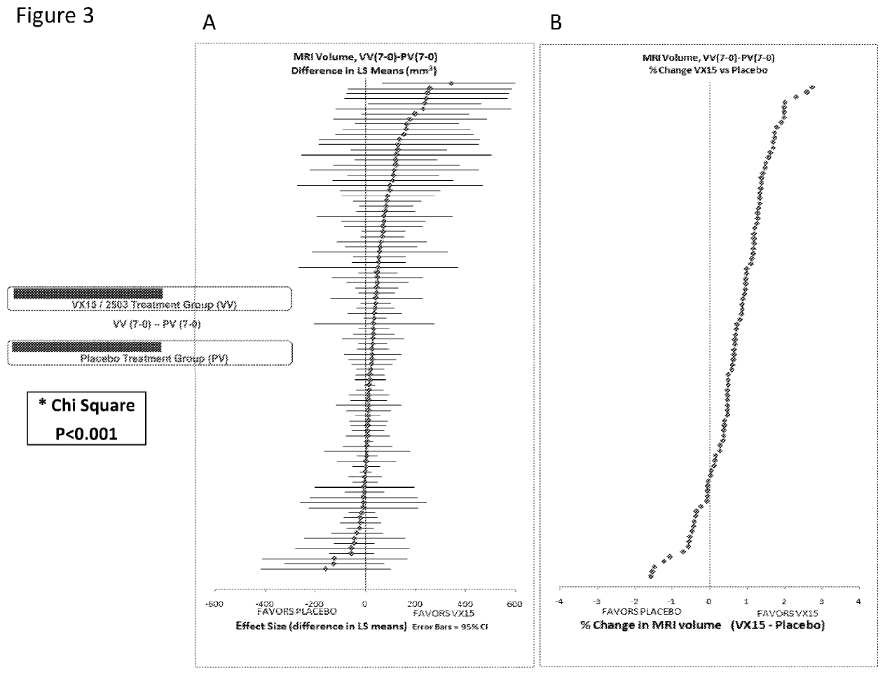

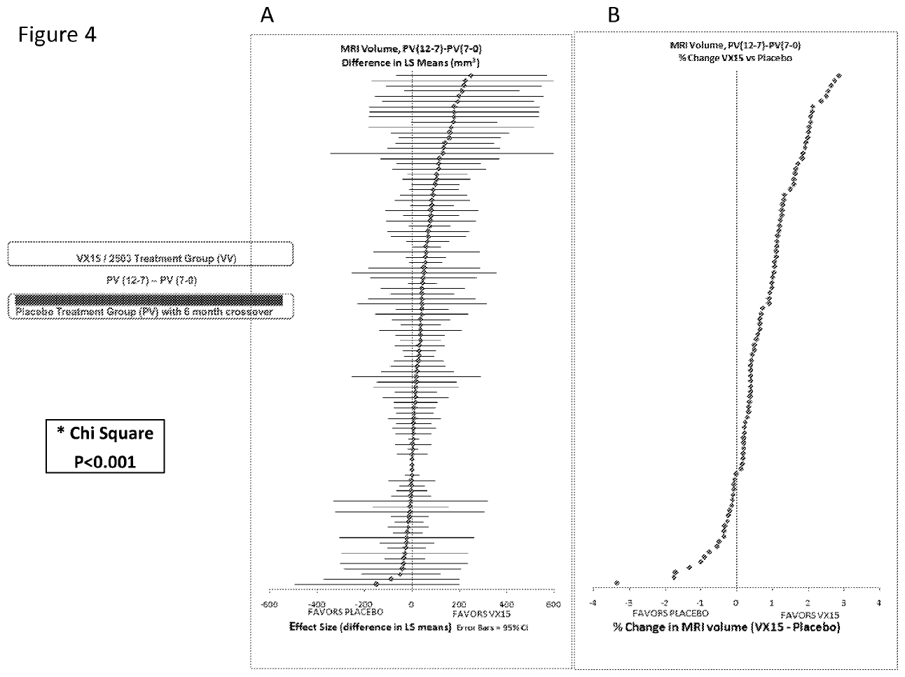

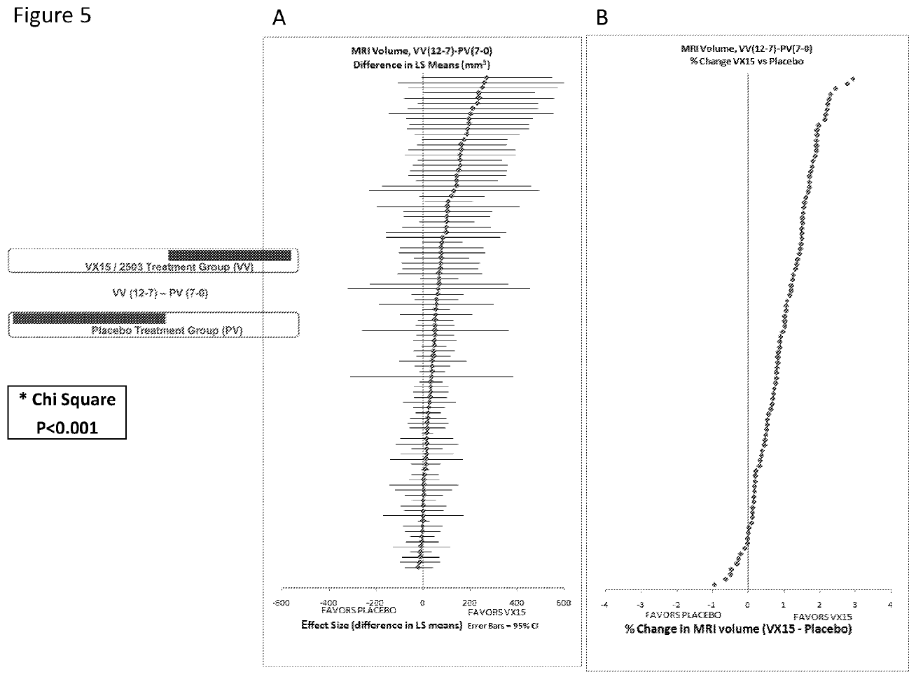

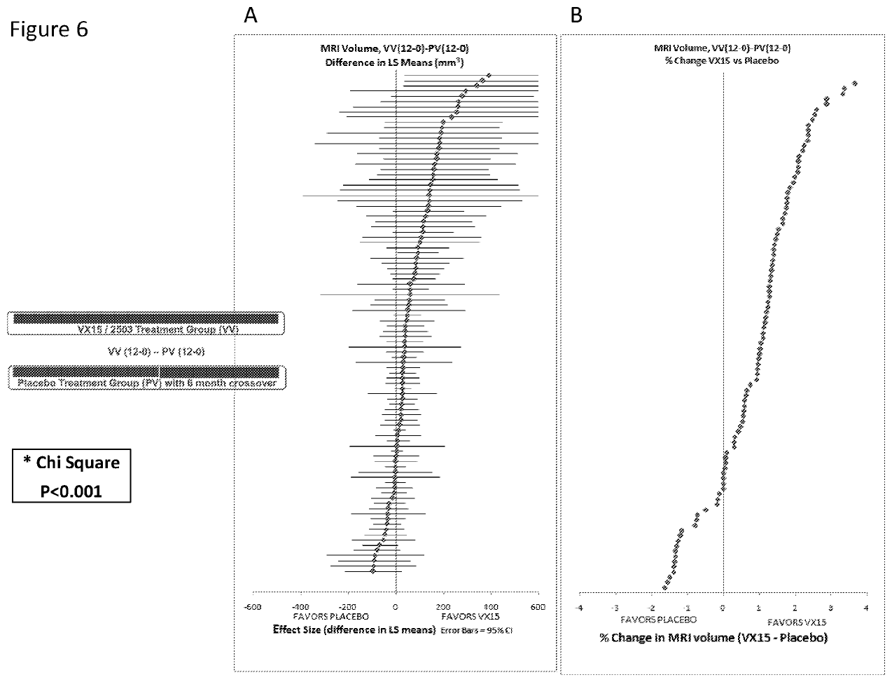

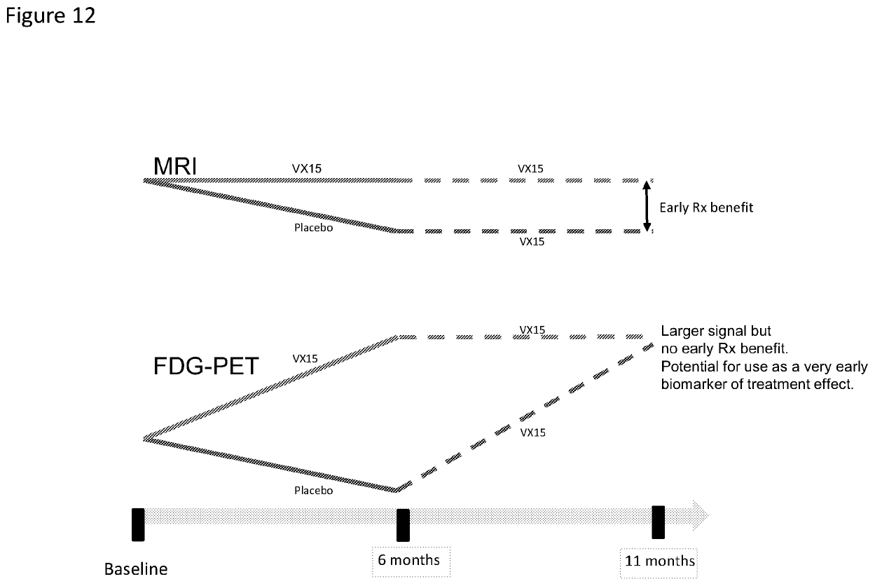

The MRI results for Cohort A are shown in FIGS. 3-6. These results show mean changes in MRI-detected volume of different brain ROI between different time points of comparable length in subjects treated with VX15 vs placebo as detailed below. MRI volume is expressed in mm³ as determined by methods that will be familiar to those of ordinary skill in the art. As compared to the reduction in MRI volume observed during the first 6 months of treatment with placebo, PV(7-0), VX15 treatment in every other 5-6 month time frame, VV(7-0), PV(12-7) and VV(12-7), prevented or minimized disease related reduction in brain volume in the majority of ROI. For each such comparison, the null hypothesis of random distribution around zero difference can be rejected with significance P<0.001 as determined by the Chi squared statistical test. In FIG. 6, the results comparing the MRI volume changes in the placebo group (PV (12-0)) that first crossed-over to VX15 treatment after 6 months, visit 7, and the group that received VX15 treatment throughout the entire period (VV (12-0)) demonstrated that, compared to the group that had received VX15 therapy for the full 11 months, delayed start of treatment in the placebo group after 6 months did not make up for the reduction in MRI volume during the first 6 months of treatment with placebo alone. This demonstrates a preventative benefit to starting treatment early and suggests that VX15 is a disease modifying therapy. This is schematically shown in the top half of FIG. 12.

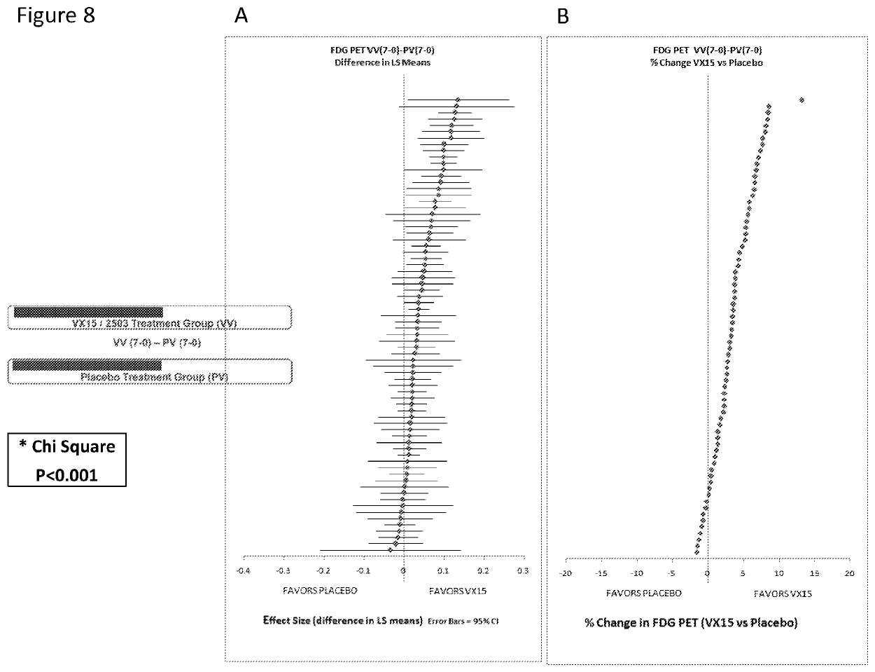

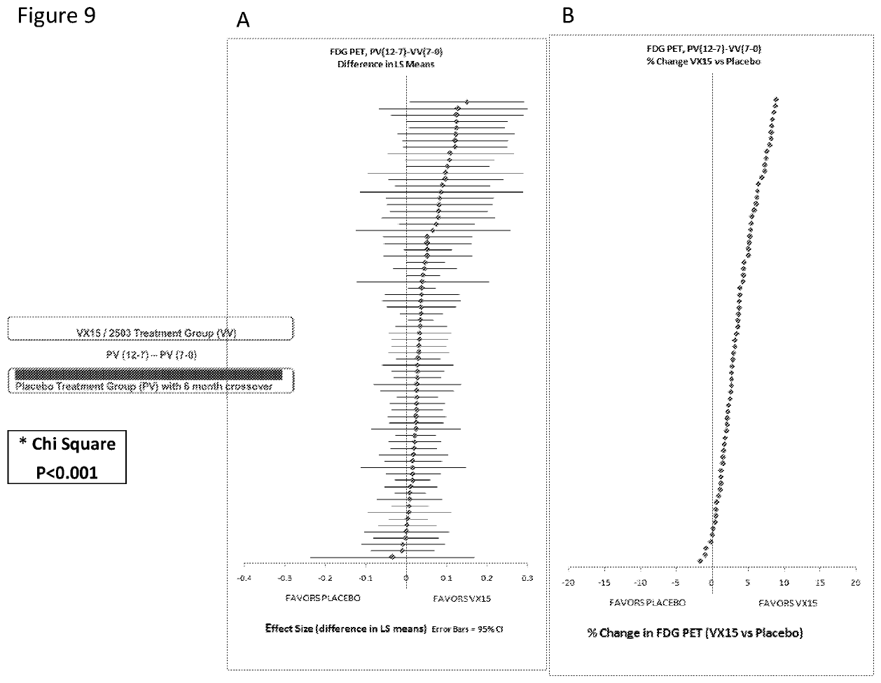

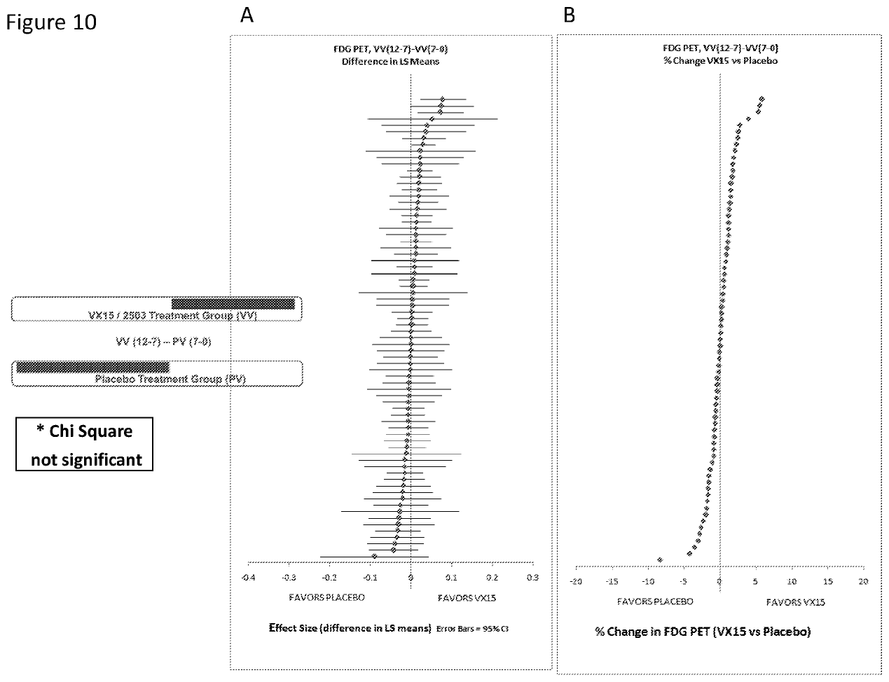

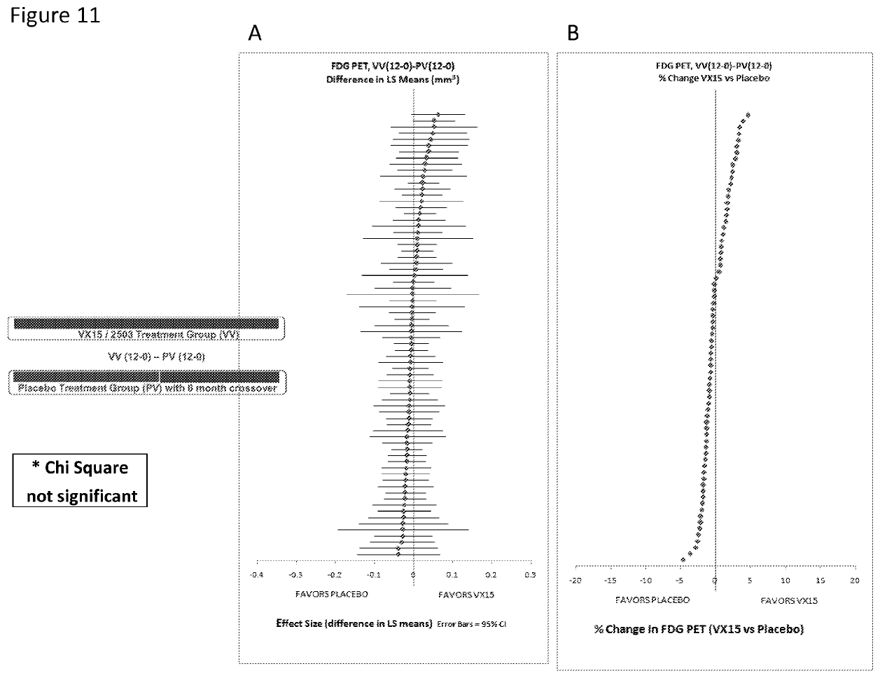

The results observed in FDG-PET imaging are shown in FIGS. 8-11. FDG-PET is expressed in SUV (Standard Uptake Values) for each brain ROI relative to a reference region, brain stem, (SUVR) for each treatment regimen and time period observed. In the first half of the trial, a supernumerary increase in glucose uptake was observed in the SEMA4D-treated (VV (7-0)) group as compared to the control placebo-treated (PV (7-0)) group (FIG. 8). That is, the mean increase in FDG-PET signal in the majority of brain ROI of the VX15-treated VV(7-0) group was greater than the mean decrease observed in the placebo PV(7-0) group for the same ROI during the same time period. Likewise, a comparison of the FDG-PET imaging of the placebo-treated patients in the first six months of treatment (PV (7-0)) and the same patients when treated with VX15 during the final 5 months of the trial (PV (12-7)), showed a supernumerary relative increase in glucose uptake (FIG. 9). In both instances, the null hypothesis of random distribution around zero difference can be rejected with p<0.001 using the chi-squared statistical test. In contrast, comparison of the mean FDG-PET signal obtained during the final five months from the patients previously treated with VX15 during the first 6 months, (VV (12-7)) with the placebo group during the first part of the trial (PV (7-0)), indicates no significant difference from a random distribution around zero by the same chi-squared test (FIG. 10). Likewise no significant difference was observed between the VX15 treated group (VV (12-0)) and the placebo-treated group (PV (12-0)) over the entire 11 months of treatment (FIG. 11).

VX15 treatment clearly resulted in a supernumerary increase in FDG-PET signal relative to the decrease observed in placebo group during the first 6 months of the treatment period whether the comparison is between VV(7-0) and PV(7-0) or PV(12-7) and PV(7-0). However, continued treatment with VX15 during the following 5 months, VV(12-7), does not repeat the same large treatment effect but appears to just prevent or minimize further decline in FDG-PET signal. Without being bound to a particular theory, a reasonable interpretation of the supernumerary benefit of initial treatment with VX15 is that it reverses an historical deficit in glucose uptake that accumulated prior to initiating treatment. It is possible that this reflects reversal of accumulated reactive astrocytes to normal astrocyte function, including increased glutamate transport and glucose uptake and glycolysis. However, once this benefit is captured by VX15 treatment during VV(7-0), it is not repeated with continued treatment during VV(12-7). The effect is schematically shown in the bottom half of FIG. 12. Observation of this correction of the historical deficit in glucose uptake can provide an early biomarker of treatment effect.

The breadth and scope of the present disclosure should not be limited by any of the above-described exemplary embodiments, but should be defined only in accordance with the following claims and their equivalents.

TABLE 6
Sequences

| SEQ | | |
|---|---|---|
| ID | | |
| NO | Description | Sequence |
| 1 | VX15/2503 | QVQLVQSGAEVKKPGSSVKVSCKASGYSFSDYYM |
| | VH | HWVRQAPGQGLEWMGQINPTTGGASYNQKFKGKA |
| | | TITVDKSTSTAYMELSSLRSEDTAVYYCARYYYG |
| | | RHFDVWGQGTTVTVSS |
| 2 | VX15/2503 | GYSFSDYYMH |
| | HCDR1 | |
| 3 | VX15/2503 | QINPTTGGASYNQKFKG |
| | HCDR2 | |
| 4 | VX15/2503 | YYYGRHFDV |
| | HCDR3 | |
| 5 | VX15/2503 | DIVMTQSPDSLAVSLGERATINCKASQSVDYDGDS |
| | VL | YMNWYQQKPGQPPKLLIYAASNLESGVPDRFSGSG |
| | | SGTDFTLTISSLQAEDVAVYYCQQSNEDPYTFGQG |
| | | TKLEIK |
| 6 | VX15/2503 | KASQSVDYDGDSYMN |
| | LCDR1 | |
| 7 | VX15/2503 | AASNLES |
| | LCDR2 | |
| 8 | VX15/2503 | QQSNEDPYT |
| | LCDR3 | |
| 9 | Mab 67 VH | QVQLQQSGPELVKPGASVKISCKASGYSFSDYYMH |
| | | WVKQSPENSLEWIGQINPTTGGASYNQKFKGKATL |
| | | TVDKSSSTAYMQLKSLTSEESAVYYCTRYYYGRHF |
| | | DVWGQGTTVTVSS |
| 10 | Mab 67 VL | DIVMTQSPASLAVSLGQRATISCKASQSVDYDGDS |
| | | YMNWYQQKPGQPPKLLIYAASNLESGIPARFSGSG |
| | | SGTDFTLNIHPVEEEDAATYYCQQSNEDPYTFGGG |
| | | TKLEIK |

What is claimed is:

1. A method for determining whether a semaphorin 4D (SEMA4D) antagonist antibody or antigen-binding fragment thereof could be effective in treating a neurodegenerative or neuroinflammatory disease, disorder, or injury, comprising:
(a) administering an effective amount of a SEMA4D antagonist antibody or antigen-binding fragment thereof to a subject having, suspected of having, or at risk of developing a neurodegenerative or neuroinflammatory disease, disorder, or injury;
(b) measuring the level of glucose uptake in the subject's brain relative to a baseline level of glucose uptake in the subject's brain measured prior to administration of the SEMA4D antagonist; and
i. continuing administration of the SEMA4D antagonist antibody or fragment thereof if an increase in glucose uptake over baseline is detected; or
ii. discontinuing administration of the SEMA4D antagonist antibody or fragment thereof if no change or a decrease in glucose uptake relative to baseline is detected.

2. A method for determining whether a semaphorin 4D (SEMA4D) antagonist antibody or antigen-binding fragment thereof will be effective in treating a neurodegenerative or neuroinflammatory disease, disorder, or injury, comprising:
(a) measuring the baseline level of glucose uptake in the brain of a subject having, suspected of having, or at risk of developing a neurodegenerative or neuroinflammatory disease, disorder, or injury;
(b) administering an effective amount of a SEMA4D antagonist antibody or antigen-binding fragment thereof to the subject;
(c) remeasuring the level of glucose uptake in the subject's brain following administration of the SEMA4D antagonist antibody or fragment thereof; and
i. continuing administration of the SEMA4D antagonist antibody or fragment thereof if an increase in glucose uptake over baseline is detected; or
ii. discontinuing administration of the SEMA4D antagonist antibody or fragment thereof if no change or a decrease in glucose uptake relative to baseline is detected.

3. A method for determining an effective dose of a semaphorin 4D (SEMA4D) antagonist antibody or fragment thereof for treating a neurodegenerative or neuroinflammatory disease, disorder, or injury, comprising:
(a) administering a starting dose of a SEMA4D antagonist antibody or antigen-binding fragment thereof to a subject having, suspected of having, or at risk of developing a neurodegenerative or neuroinflammatory disease, disorder, or injury;
(b) measuring the level of glucose uptake in the subject's brain relative to a baseline level of glucose uptake in the subject's brain measured prior to administration of the SEMA4D antagonist; and
i. adjusting the dose of the SEMA4D antagonist antibody or fragment thereof if an increase in glucose uptake over baseline is detected, the adjustment determined on the level of increase, or
ii. discontinuing administration of the SEMA4D antagonist antibody or fragment thereof if no change or a decrease in glucose uptake relative to baseline is detected.

4. The method of claim 3, further comprising increasing the dose of SEMA4D antagonist antibody relative to that tested in step (b) and remeasuring the change in level of glucose uptake relative to a new baseline in a different previously untreated patient or in the same patient following withdrawal of treatment in the same patient for a period of time determined to allow accumulation of a historical deficit in that neurodegenerative or neuroinflammatory disease, disorder, or injury, and further adjusting the dose of the SEMA4D antagonist antibody if a further increase is detected.

5. A method for determining an effective dose of a semaphorin 4D (SEMA4D) antagonist antibody or fragment thereof for treating a neurodegenerative or neuroinflammatory disease, disorder, or injury, comprising:
(a) measuring the baseline level of glucose uptake in the brain of a subject having, suspected of having, or at risk of developing a neurodegenerative or neuroinflammatory disease, disorder, or injury;
(b) administering a starting dose of a SEMA4D antagonist antibody or antigen-binding fragment thereof to the subject;
(c) remeasuring the level of glucose uptake in the subject's brain following administration of the SEMA4D antagonist antibody or fragment thereof; and
i. adjusting the dose of the SEMA4D antagonist antibody or fragment thereof if an increase in glucose uptake over baseline is detected, the adjustment determined on the level of increase, or
ii. discontinuing administration of the SEMA4D antagonist antibody or fragment thereof if no change or a decrease in glucose uptake relative to baseline is detected.

6. The method of claim 5, further comprising increasing the dose of SEMA4D antagonist antibody relative to that tested in step (c) and remeasuring the change in level of glucose uptake relative to a new baseline in a different previously untreated patient or in the same patient following withdrawal of treatment in the same patient for a period of time determined to allow accumulation of a historical deficit in that neurodegenerative or neuroinflammatory disease, disorder, or injury, and further adjusting the dose of the SEMA4D antagonist antibody if a further increase is detected.

7. A method for treating a subject having, suspected of having, or at risk of developing a neurodegenerative or neuroinflammatory disease, disorder, or injury, comprising:
(a) administering a SEMA4D antagonist antibody or antigen-binding fragment thereof to a subject having, suspected of having, or at risk of developing a neurodegenerative or neuroinflammatory disease, disorder, or injury;
(b) measuring the level of glucose uptake in the subject's brain relative to a baseline level of glucose uptake in the subject's brain measured prior to administration of SEMA4D antagonist; and
i. continuing administration of the SEMA4D antagonist antibody or fragment thereof if an increase in glucose uptake over baseline is detected;
or
ii. discontinuing administration of the SEMA4D antagonist antibody or fragment thereof if no change or a decrease in glucose uptake relative to baseline is detected.

8. A method for treating a subject having, suspected of having, or at risk of developing a neurodegenerative or neuroinflammatory disease, disorder, or injury, comprising:
(a) measuring the baseline level of glucose uptake in the brain of a subject having, determined to have, or suspected of having a neurodegenerative or neuroinflammatory disease, disorder, or injury;
(b) administering a SEMA4D antagonist antibody or antigen-binding fragment thereof to the subject;
(c) remeasuring the level of glucose uptake in the subject's brain following administration of the SEMA4D antagonist antibody or fragment thereof; and
i. continuing administration of the SEMA4D antagonist antibody or fragment thereof if an increase in glucose uptake over baseline is detected;
or
ii. discontinuing administration of the SEMA4D antagonist antibody or fragment thereof if no change or a decrease in glucose uptake relative to baseline is detected.

9. A method for determining whether a semaphorin 4D (SEMA4D) antagonist antibody or antigen-binding fragment thereof will be effective in treating a neurodegenerative or neuroinflammatory disease, disorder, or injury, comprising:
(a) administering a SEMA4D antagonist antibody or antigen-binding fragment thereof to a subject having, suspected of having, or at risk of developing a neurodegenerative or neuroinflammatory disease, disorder, or injury;
(b) ordering the measurement of the level of glucose uptake in the subject's brain relative to a baseline level of glucose uptake in the subject's brain measured prior to administration of the SEMA4D antagonist; and
i. continuing administration of the SEMA4D antagonist antibody or fragment thereof if an increase in glucose uptake over baseline is detected;
or
ii. discontinuing administration of the SEMA4D antagonist antibody or fragment thereof if no change or a decrease in glucose uptake relative to baseline is detected.

10. A method for determining whether a semaphorin 4D (SEMA4D) antagonist antibody or antigen-binding fragment thereof will be effective in treating a neurodegenerative or neuroinflammatory disease, disorder, or injury, comprising:
(a) ordering the measurement of a baseline level of glucose uptake in the brain of a subject having, suspected of having, or at risk of developing a neurodegenerative or neuroinflammatory disease, disorder, or injury;
(b) administering a SEMA4D antagonist antibody or antigen-binding fragment thereof to the subject;
(c) ordering remeasurement of the level of glucose uptake in the subject's brain following administration of the SEMA4D antagonist antibody or fragment thereof, and
i. continuing administration of the SEMA4D antagonist antibody or fragment thereof if an increase in glucose uptake over baseline is detected;
or
ii. discontinuing administration of the SEMA4D antagonist antibody or fragment thereof if no change or a decrease in glucose uptake relative to baseline is detected.

11. A method for determining whether a semaphorin 4D (SEMA4D) antagonist antibody or antigen-binding fragment thereof will be effective in treating a neurodegenerative or neuroinflammatory disease, disorder, or injury, comprising:
(a) measuring the baseline level of glucose uptake in the brain of a subject presented as having, suspected of having, or at risk of developing a neurodegenerative or neuroinflammatory disease, disorder, or injury; and
(b) remeasuring the level of glucose uptake in the subject's brain following administration or a SEMA4D antagonist antibody or antigen-binding fragment thereof to the subject by a healthcare provider; and
i. instructing the healthcare provider to continue administration of the SEMA4D antagonist antibody or fragment thereof if an increase in glucose uptake over baseline is detected; or
ii. instructing the healthcare provider to discontinue administration of the SEMA4D antagonist antibody or fragment thereof if no change or a decrease in glucose uptake relative to baseline is detected.

12. The method of any one of claims 1 to 11, wherein the SEMA4D antagonist antibody or fragment thereof inhibits SEMA4D interaction with its receptor.

13. The method of claim 12, wherein the receptor is Plexin-B1, Plexin-B2, or CD72.

14. The method of any one of claims 1-13, wherein the SEMA4D antagonist antibody or fragment thereof inhibits SEMA4D-mediated Plexin-B1 signal transduction.

15. The method of any one of claims 1-14, wherein the SEMA4D antagonist antibody or fragment thereof competitively inhibits a reference antibody comprising a variable heavy chain region (VH) comprising the amino acid sequence SEQ ID NO: 1 and a variable light chain region (VL) comprising the amino acid sequence SEQ ID NO: 5 from binding to SEMA4D.

16. The method of any one of claims 1 to 15, wherein the SEMA4D antagonist antibody or fragment thereof binds to the same SEMA4D epitope as a reference antibody comprising a VH comprising the amino acid sequence SEQ ID NO: 1 and a VL comprising the amino acid sequence SEQ ID NO: 5.

17. The method of claim 15 or claim 16, wherein the SEMA4D antagonist antibody comprises a VH and a VL, wherein the VH comprises three complementarity determining regions (CDRs) HCDR1, HCDR2, and HCDR3, wherein the VL comprises three CDRs LCDR1, LCDR2, and LCDR3, and wherein the CDRs comprise the amino acid sequences SEQ ID NO: 2, SEQ ID NO: 3, SEQ ID NO: 4, SEQ ID NO: 6, SEQ ID NO: 7, and SEQ ID NO: 8, respectively except for at least one or two single conservative amino acid substitutions in one or more of the CDRs.

18. The method of claim 15 or claim 16, wherein the SEMA4D antagonist antibody comprises a VH and a VL, wherein the VH comprises three complementarity determining regions (CDRs) HCDR1, HCDR2, and HCDR3, wherein the VL comprises three CDRs LCDR1, LCDR2, and LCDR3, and wherein the CDRs comprise the amino acid sequences SEQ ID NO: 2, SEQ ID NO: 3, SEQ ID NO: 4, SEQ ID NO: 6, SEQ ID NO: 7, and SEQ ID NO: 8, respectively.

19. The method of any one of claims 15 to 18, wherein the VH comprises an amino acid sequence at least 90% identical to SEQ ID NO: 1 and the VL comprises an amino acid sequence at least 90% identical to SEQ ID NO: 5.

20. The method of claim 19, wherein the VH comprises the amino acid sequence SEQ ID NO: 1 and the VL comprises the amino acid sequence SEQ ID NO: 5.

21. The method of any one of claims 1 to 20, wherein a first dose of the SEMA4D antagonist antibody is administered, and then the SEMA4D antagonist antibody is administered at least once every week, at least once every two weeks, at least once every three weeks, at least once a month, or at least once every two months thereafter.

22. The method of claim 21, wherein the baseline measurement of glucose uptake is measured just prior to the first dose of the SEMA4D antagonist antibody.

23. The method of claim 21 or claim 22 wherein the change in glucose uptake relative to baseline is measured at least one week after the first dose, at least two weeks after the first dose, at least one month after the first dose, at least two months after the first dose, at least three months after the first dose, at least four months after the first dose, at least five months after the first dose, at least six months after the first dose, or any combination thereof.

24. The method of any one of claims 1 to 23, wherein glucose uptake in the subject's brain is measured by 18F-Fluorodeoxyglucose Positron Emission Tomography (FDG-PET) imaging.

25. The method of any one of claims 1 to 24, wherein the subject is a human.

26. The method of any one of claims 1 to 25, wherein the neurodegenerative or neuroinflammatory disease, disorder or injury is Alzheimer's disease, Parkinson's disease, Huntington's disease, Down syndrome, ataxia, amyotrophic lateral sclerosis (ALS), multiple sclerosis, (MS), epilepsy, meningitis, brain edema, spinal cord injury, traumatic brain injury, frontotemporal dementia (FTD), HIV-related cognitive impairment, CNS Lupus, mild cognitive impairment, or a combination thereof.

27. The method of claim 26 wherein the neurodegenerative or neuroinflammatory disease, disorder or injury is Huntington's disease (HD).

28. The method of claim 27, wherein the subject is at risk of developing HD due to familial history of HD or genetic testing.

29. The method of claim 28, wherein genetic testing reveals 36 or more CAG repeats in the subject's HTT gene.

30. The method of any one of claims 27 to 29, wherein the subject is suspected of having HD due to mild motor dysfunction, mild cognitive impairment, or mild neuropsychiatric features.

31. The method of any one of claims 27 to 30, wherein the subject is diagnosed as having HD due to brain atrophy, an elevated Uniform Huntington's Disease Rating Scale score (UHDRS), an increased Huntington's Disease Cognitive Assessment Battery (HD-CAB) score, an increased Huntington's Disease Quantitative Motor Assessment score or a combination thereof.

32. The method of claim 31, wherein the subject is in the presymptomatic, early prodromal, late prodromal, early manifest, moderate manifest, or advanced manifest stage of HD.
